(19) United States
(12) Patent Application Publication
(10) Pub. No.: US 2013/0345086 A1
(43) Pub. Date: Dec. 26, 2013

(54) CD4+ T-CELL GENE SIGNATURE FOR RHEUMATOID ARTHRITIS (RA)

(71) Applicants: John D. Isaacs, Newcastle upon Tyne (GB); Arthur G. Pratt, Newcastle upon Tyne (GB)

(72) Inventors: John D. Isaacs, Newcastle upon Tyne (GB); Arthur G. Pratt, Newcastle upon Tyne (GB)

(73) Assignee: UNIVERSITY OF NEWCASTLE UPON TYNE, Newcastle upon Tyne (GB)

(21) Appl. No.: 13/985,249

(22) PCT Filed: Feb. 13, 2012
(86) PCT No.: PCT/GB2012/050315

§ 371 Date: Sep. 11, 2013

(30) Foreign Application Priority Data
Feb. 14, 2011 (GB) 1102563.2
May 25, 2011 (GB) 1108818.4

(51) Int. Cl. C12Q 1/68

(52) U.S. Cl. CPC C12Q 1/6883
USPC 506/9; 435/7.1; 506/16

(57) ABSTRACT

The present invention relates to methods and products for the identification and diagnosis of Rheumatoid arthritis (RA), in particular for the diagnosis of anti-citrullinated peptide antibody (ACPA)-negative RA. Most particularly the invention relates to a gene expression signature comprising at least 12 biomarkers for use in the prognosis or diagnosis of RA.

The present invention relates to methods and products for the identification and diagnosis of Rheumatoid arthritis (RA), in particular for the diagnosis of anti-citrullinated peptide antibody (ACPA)-negative RA. Most particularly the invention relates to a gene expression signature comprising 12 biomarkers for use in the prognosis or diagnosis of RA.

Rheumatoid arthritis (RA) is a chronic, disabling autoimmune disease with a predilection for peripheral joints(1). The importance of prompt disease-modifying therapy in improving clinical outcomes is reinforced by international management guidelines(2). However, approximately 40% of patients with new-onset inflammatory arthritis have disease which is unclassifiable at inception, and are said to have an undifferentiated arthritis (UA)(3). Recently, a validated “prediction rule” has been developed for use amongst UA patients, whereby a composite score derived from clinical and serological data predicts risk of progression to RA(4). The scoring system relies heavily on autoantibody and, in particular, anti-citrullinated peptide antibody (ACPA) status, highlighting the specificity of circulating ACPA for RA(5). However, the diagnosis of ACPA-negative RA remains challenging in the early arthritis clinic, being frequently delayed despite application of the prediction rule(6).

Technological and computational advances have permitted high-throughput, “discovery-driven” routes to biomarker identification in clinical settings through whole-genome transcription profiling(7). Transcriptome analysis in RA has usually been limited to cross-sectional comparisons with normal controls(8, 9), with exceptions aiming to predict responsiveness to biologic agents in established disease(10). Recent work has demonstrated the potential for peripheral blood mononuclear cells (PBMCs) to yield clinically relevant prognostic “gene signatures” in autoimmune disease(11). The application of a similar, prospective, approach to the discovery of predictive biomarkers in UA should compliment existing diagnostic algorithms, whilst providing new insights into disease pathogenesis(12). However, the use of PBMC for transcriptional analysis may result in data that are biased by relative subset abundance (13). To address this, protocols for the rapid ex vivo positive selection of subsets for the purpose of transcription profiling have been validated(14), permitting scrutiny of pathophysiologically relevant cells in isolation.

Although no single cell-type is exclusively implicated in RA, many of the established and emerging genetic associations of the condition implicate the CD4+ T-cell as a key player, and anomalies in peripheral blood CD4+ T-cell phenotype are well-documented(15, 16). For example, in addition to the long-recognised association of the disease with particular MHC class II alleles that encode a conserved sequence within the peptide binding groove (“shared epitope”)(12), recent genome-wide association scans have implicated protein tyrosine phosphatase 22 (involved in T-cell receptor signalling), the IL2-receptor, the co-stimulatory molecules CD28, CTLA-4 and CD40, and the potentially lineage-defining signal transduction and activator of transcription 4 (STAT4) molecules(17). The inventors have therefore surmised that the peripheral blood (PB) CD4+ T-cell transcriptome might therefore represent a plausible substrate for predictive biomarker discovery in early arthritis.

The following terms are used throughout this document.

A sample is any biological material obtained from an individual.

A polynucleotide is a polymeric form of nucleotides of any length. Nucleotides can be either ribonucleotides or deoxyribonucleotides. The term covers, but is not limited to, single-, double-, or multi-stranded deoxyribonucleic acid (DNA) or ribonucleic acid (RNA), mRNA, genomic DNA, cDNA, DNA-RNA hybrids, or a polymer comprising natural, chemically or biochemically modified, non-natural nucleotide bases. Such polynucleotides may include modifications such as those required to allow attachment to a solid support.

A gene is a polynucleotide sequence that comprises sequences that are expressed in a cell as RNA and control sequences necessary for the production of a transcript or precursor.

A gene expression product can be encoded by a full length coding sequence or by any portion of the coding sequence.

A probe can be a DNA molecule such as a genomic DNA or fragment thereof, an RNA molecule, a cDNA molecule or fragment thereof, a PCR product, a synthetic oligonucleotide, or any combination thereof. Said probe can be a derivative or variant of a nucleic acid molecule, such as, for example, a peptide nucleic acid molecule.

A probe can be specific for a target when it comprises a continuous stretch of nucleotides that are entirely complementary to a target nucleotide sequence (generally an RNA product of said gene, or a cDNA product thereof). However a probe can also be considered to be specific if it comprises a continuous stretch of nucleotides that are partially complementary to a target nucleotide sequence. Partially in this instance can be taken to mean that a maximum of 10% from the nucleotides in a continuous stretch of at least 20 nucleotides differs from the corresponding nucleotide sequence of a RNA product of said gene. The term complementary is well known and refers to a sequence that is related by base-pairing rules to the target sequence. Probes will generally be designed to minimise non-specific hybridization.

Where reference is made to “one or more” or “12 or more” or “X or more” genes, this can be understood to be for the purposes of illustration and are non-limiting (although may illustrate best or preferred options).

Gene-lists 1-9 as referenced in the following description are provided at the end of the description. In lists 2-5, the “Illumina ID” column contains the probe address number on the Illumina WG6 (v3) BeadChip (http://www.illumina.com/support/annotation_files.ilmn). Where >1 “differentially expressed” Illumina probes appearing in a given list corresponded to a single gene entity, duplicates were removed. Uncorrected p-values are given in lists 2-5. Official gene symbols and RefSeq accession numbers are given for identification purposes. Non-linearised fold-change values are given values >1 indicate genes up-regulated in RA relative to non-RA groups in any given comparison. (Values <1=down-regulated). For down-regulated values, linearised data may be obtained by rendering the negative reciprocal of the non-linearised value; i.e FC of 0.75=>|FC| of −1.33. Gene lists are ranked according to FC. Any additional list-specific information is provided on the relevant pages.

In order to provide further clarity to the reader, certain sequences are provided in full herein. In particular, the following sequence data is referred to herein;

| Gene | Accession No | Sequence ID |
|---|---|---|
| BCL3 | NM_005178.2 | SEQ ID No 1 |
| SOCS3 | NM_003955.3 | SEQ ID No 2 |
| PIM1 | NM_002648.2 | SEQ ID No 3 |
| SBNO2 | NM_014963.2 | SEQ ID No 4 |
| LDHA | NM_005566.1 | SEQ ID No 5 |
| CMAH | NR_002174.2 | SEQ ID No 6 |
| NOG | NM_005450.2 | SEQ ID No 7 |
| PDCD1 | NM_005018.1 | SEQ ID No 8 |
| IGFL2 | NM_001002915.1 | SEQ ID No 9 |
| LOC731186 | XM_001128760.1 | SEQ ID No 10 |
| MUC1 | NM_001044391.1 | SEQ ID No 11 |
| GPRIN3 | CR743148 | SEQ ID No 12 |
| CD40LG | NM_000074.2 | SEQ ID No 13 |

According to the present invention there is provided a method of diagnosing Rheumatoid arthritis in a patient, the method comprising:

obtaining a sample comprising CD4+ T-cells from the patient; and
determining expression levels of one or more genes selected from the group consisting of

### BCL3

### SOCS3

### PIM1

### SBNO2

### LDHA

### CMAH

### NOG

### PDCD1

### IGFL2

### LOC731186

### MUC1

### GPRIN3; and

comparing said expression levels to reference expression levels, wherein a difference in expression of said one or more genes indicates an increased likelihood that the patient has Rheumatoid arthritis.

Optionally the group further consists of CD40LG.

Generally the reference expression levels are representative of levels found in samples comprising cells from a patient who does not have RA.

It has been found that an increase in expression when compared to the reference expression levels indicates an increased likelihood that the patient has rheumatoid arthritis.

The inventors' work has confirmed the utility of the signature where CD4+ T-cells of >95% purity are used, and preliminary data suggest that there is some overlap where whole blood RNA (from unpurified cells) is used as substrate.

Most preferably the step of determining expression levels of one or more genes selected from the group consisting of

### BCL3

### SOCS3

### PIM1

### SBNO2

### LDHA

### CMAH

### NOG

### PDCD1

### IGFL2

### LOC731186

### MUC1

### GPRIN3

includes determining expression levels for all of the genes from the group.

The group may be referred to as a “12 gene signature”

Optionally the group further comprises the gene CD40LG.

This group may be referred to as a “13 gene signature”

It has been shown that a difference in expression when compared to the reference expression levels of all of said one or more genes indicates an increased likelihood that the patient has Rheumatoid arthritis

According to the present invention there is provided an in vitro method for typing a sample from an individual classified as having undifferentiated arthritis, or suspected to suffer from rheumatoid arthritis, the method comprising:

obtaining a sample from the individual; and
determining expression levels of one or more genes selected from the group consisting of

### BCL3

### SOCS3

### PIM1

### SBNO2

### LDHA

### CMAH

### NOG

### PDCD1

### IGFL2

### LOC731186

### MUC1

### GPRIN3; and

typing said sample on the basis of the expression levels determined; wherein said typing provides prognostic information related to the risk that the individual has rheumatoid arthritis (RA).

Optionally the group further comprises the gene CD40LG.

Most preferably expression levels are determined by determining RNA levels.

Methods for determining mRNA levels are well established, some being described herein.

Preferably the sample comprises CD4+ T cells.

Preferably the sample is peripheral whole blood.

Preferably the methods include the step of separating CD4+ T cells from peripheral whole blood.

Preferably the methods include extracting RNA from the CD4+ T cells.

Most preferably the method is for diagnosing anti-citrullinated peptide antibody (ACPA)-negative rheumatoid arthritis.

Preferably expression levels of all of the genes in the group are determined and compared to a set of reference expression levels.

Optionally the method further comprises the step of combining the results of the 12 gene signature with the results of known prediction analysis. The 13 signature could be used instead of the 12 gene signature.

Preferably the known prediction analysis is the Leiden prediction rule (Reference; van der Helm-van Mil 2008 Arthritis and Rheumatism_{—}

Using a composite of the 12 gene signature (or 13 gene signature)/Leiden prediction test maximises the specificity, precision and sensitivity of the test.

According to another aspect of the present invention there is provided a method of diagnosing rheumatoid arthritis in a patient, the method comprising:

obtaining a blood sample from the patient; and
determining expression/mRNA levels of 12 or more genes selected from the group defined in GENE LIST 2; and
comparing said expression/mRNA levels to a set of reference expression/mRNA levels, wherein a difference in expression of said 12 or more genes indicates an increased likelihood that the patient has Rheumatoid arthritis.

According to another aspect of the present invention there is provided a method of diagnosing Rheumatoid arthritis in a patient, the method comprising:

obtaining a blood sample from the patient; and
determining levels of Interleukin-6 (IL-6); and
comparing said levels to a set of reference IL-6 levels, wherein an difference in expression of IL-6 indicates an increased likelihood that the patient has Rheumatoid arthritis.

It has been found that an increase in expression of IL-6 indicates an increased likelihood that the patient has Rheumatoid arthritis.

Notably, serum IL-6 is notoriously sensitive to, for example, diurnal variation, and the inventors identified that it is useful to standardise the sampling procedure—all the samples were taken between the hours of 1300 and 1630, and frozen to −80 within 4 hours of blood draw, undergoing no more than 1 freeze-thaw cycle, for example.

Most preferably the method is for diagnosing anti-citrullinated peptide antibody (ACPA)-negative rheumatoid arthritis.

Preferably the results of the IL-6 expression analysis are combined with the results of known prediction analysis.

An array comprising (a) a substrate and (b) 12 or more different elements, each element comprising at least one polynucleotide that binds to a specific mRNA transcript, said mRNA transcript being of a gene selected from the group defined in GENE LIST 2.

An array comprising (a) a substrate and (b) one or more different elements, each element comprising at least one polynucleotide that binds to a specific mRNA transcript, said mRNA transcript being of a gene selected from the group comprising

### BCL3

### SOCS3

### PIM1

### SBNO2

### LDHA

### CMAH

### NOG

### PDCD1

### IGFL2

### LOC731186

### MUC1

### GPRIN3

Optionally the group further comprises the gene CD40LG.

An array comprising (a) a substrate and (b) 12 elements, each element comprising at least one polynucleotide that binds to an mRNA transcript, said array comprising a binding element for the mRNA of each of the following group of genes

### BCL3

### SOCS3

### PIM1

### SBNO2

### LDHA

### CMAH

### NOG

### PDCD1

### IGFL2

### LOC731186

### MUC1

### GPRIN3

Optionally the array further comprises an additional element comprising at least one polynucleotide that binds to an mRNA transcript for CD40LG.

Preferably the substrate is a solid substrate,

A kit comprising an array as described above and instructions for its use.

Use of a set of probes comprising polynucleotides specific for 12 or more of the genes listed in GENE LIST 2.

Use of a set of probes comprising polynucleotides specific for one or more of the genes selected from the list;

### BCL3

### SOCS3

### PIM1

### SBNO2

### LDHA

### CMAH

### NOG

### PDCD1

### IGFL2

### LOC731186

### MUC1

### GPRIN3

for determining the risk of an individual suffering from rheumatoid arthritis.

Optionally the set of probes further comprises a polynucleotide specific for CD40LG.

Use of a set of probes comprising polynucleotides specific for the genes selected from the list;

### BCL3

### SOCS3

### PIM1

### SBNO2

### LDHA

### CMAH

### NOG

### PDCD1

### IGFL2

### LOC731186

### MUC1

### GPRIN3

for determining the risk of an individual suffering from rheumatoid arthritis.

Optionally the set of probes further comprises a polynucleotide specific for CD40LG.

Use of a set of probes comprising primers specific for one or more of the genes selected from the list;

### BCL3

### SOCS3

### PIM1

### SBNO2

### LDHA

### CMAH

### NOG

### PDCD1

### IGFL2

### LOC731186

### MUC1

### GPRIN3

for determining the risk of an individual suffering from rheumatoid arthritis.

Optionally the set of probes further comprises a primer specific for CD40LG.

Use of a set of probes comprising primers specific for the genes selected from the list;

### BCL3

### SOCS3

### PIM1

### SBNO2

### LDHA

### CMAH

### NOG

### PDCD1

### IGFL2

### LOC731186

### MUC1

### GPRIN3

for determining the risk of an individual suffering from rheumatoid arthritis.

Optionally the set of probes further comprises a primer specific for CD40LG.

Most preferably the use of a set of probes is for determining the risk of an individual suffering from anti-citrullinated peptide antibody (ACPA)-negative rheumatoid arthritis.

According to a further aspect of the present invention there is provided an IL-6 receptor blocker for the treatment of RA.

This kind of biomarker would be expected to have utility in stratifying early RA patients into subgroups of therapeutic significance. For example, patients with high baseline IL-6 (and potentially also relatively highly dysregulated STAT3-inducible genes in circulating CD4+ T-cells, as a consequence), could potentially be more effectively be managed using an IL-6 signalling blocker (such as tocilizumab) or a Jak1/3 inhibitor.

Optionally the IL-6 receptor blocker is tocilizumab.

Optionally the IL-6 receptor blocker is a Jak1/3 inhibitor.

In order to provide a better understanding of the present invention further details and examples will be provided below with reference to the following figures and tables;

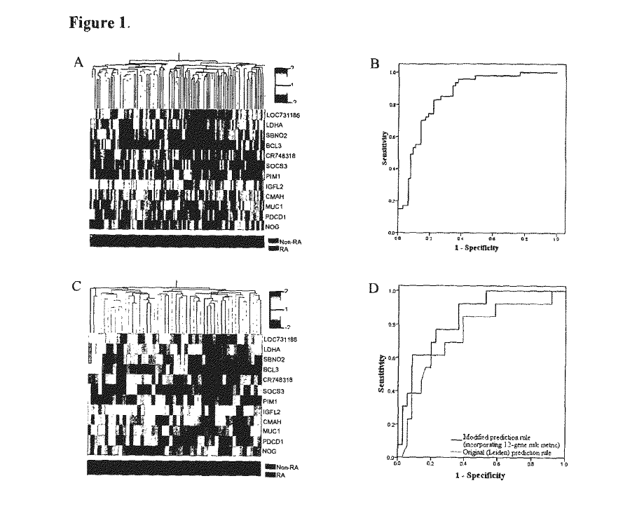

FIG. 1. Peripheral blood CD4+ T-cell expression of 12-gene signature is discriminatory for early RA. A. Hierarchical clustering of training-set samples based on similarity in gene expression. 111 samples are represented by columns and indicated individual genes by rows; the colour at each co-ordinate indicates gene-wise fold-expression relative to median, according to the colour scale to the right of the figure. Underlying colour-bar labels samples by inception diagnosis, confirmed in each case at >1 year follow-up. B. ROC plot from a range of cut-offs for an RA risk metric derived from normalised gene expression values in training cohort (see text). Area under curve=0.85; Standard error of the mean=0.04; p<0.001. C. Hierarchical clustering of validation UA sample set based on correlations in expression patterns of the same genes (interpretation as for FIG. 1A). D. ROC curves comparing discriminatory value of original Leiden prediction rule (grey line) with a modified metric incorporating 12-gene signature (see text). The modified metric confers added value to the original Leiden prediction score: AU ROC curve (original Leiden prediction rule)=0.74; SEM=0.08, versus AU ROC curve (modified metric incorporating gene signature)=0.84; SEM=0.06. p<0.001 in both cases.

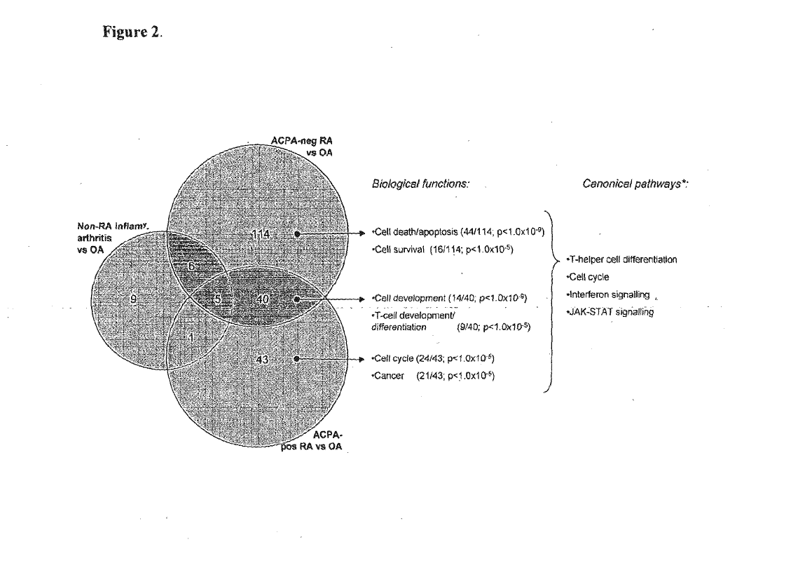

FIG. 2. Functional analysis of array data. Non-redundant lists of genes differentially expressed (>1.2 fold-change; p<0.05) between OA and 3 separate inflammatory comparator groups were overlapped in a Venn-diagram (see text, and Gene-lists 2-4 for detailed list compositions). Genes uniquely de-regulated in RA (ACPA-negative, ACPA-positive or both) could thereby be identified and subjected to pathway analysis using IPA software. The top 2 over-represented biological functions identified for the 3 indicated sets are shown, along with the proportion of the set associated with the function in question, and a p-value relating to the likelihood of given proportions occurring by chance (Fisher's exact test). Gene-lists 5-7 summarise functionally related genes thereby identified. The 3 indicated sets were combined to identify canonical pathways over-represented amongst genes differentially expressed between RA and OA in general. Pathways of particular interest in the biological context are listed (genes in question are listed in Gene-list 8), *hypergeometric p-values (Fisher's exact) in each case <0.01.

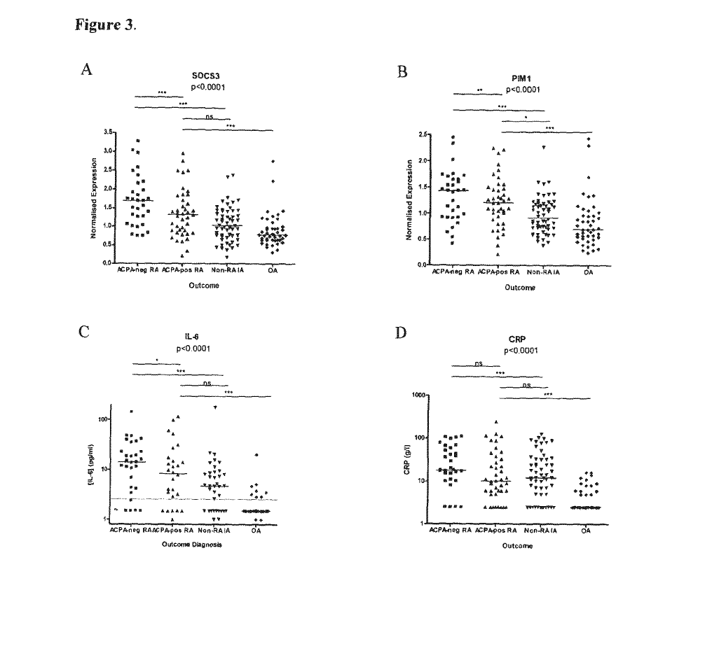

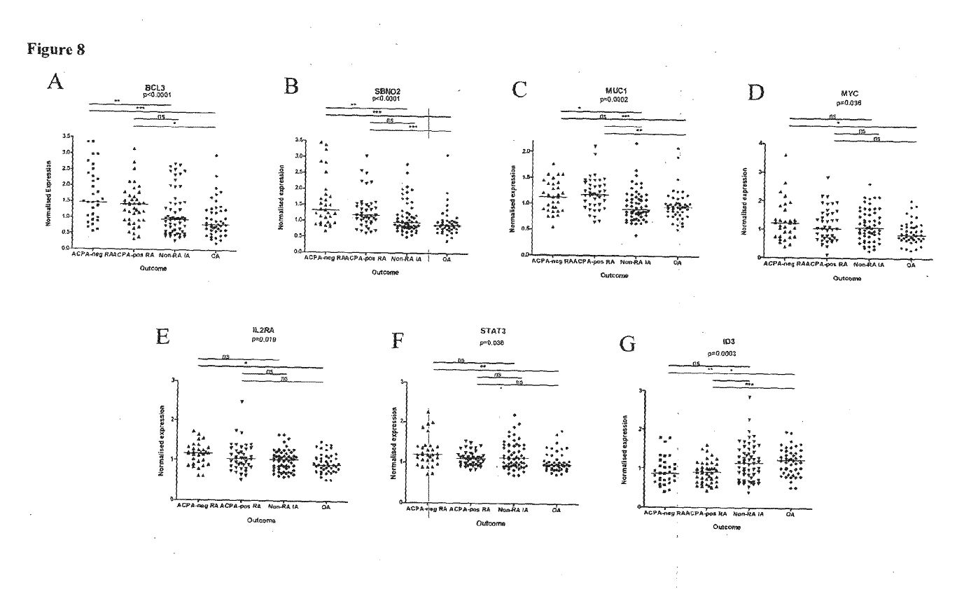

FIG. 3. A-B. PB CD4+ T-cell expression profiles of indicated STAT3-regulated genes across 4 comparator groups; see FIG. 8 for additional examples, and Table 6 for characteristics of comparator groups). C. Comparison of serum IL-6 measurements, where available, between comparator groups (n=131). Where ELISA readout was <2.6 pg/ml detection threshold (dotted line), an arbitrary value of 1.5 pg/ml was recorded. D. Comparison of CRP measurements between comparator groups (n=173). Where read-out was <5 an arbitrary value of 2.5 was recorded. A-F. P-values shown are derived from non-parametric analysis of variance (Kruskall-Wallis); for post-hoc analyses, 1, 2 and 3 asterisks denote p<0.05, 0.01 and 0.001 respectively (Dunn's multiple comparison analysis).

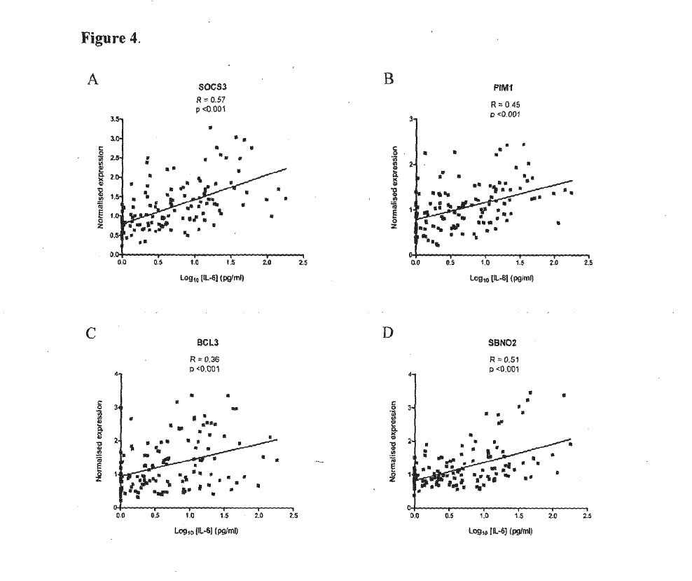

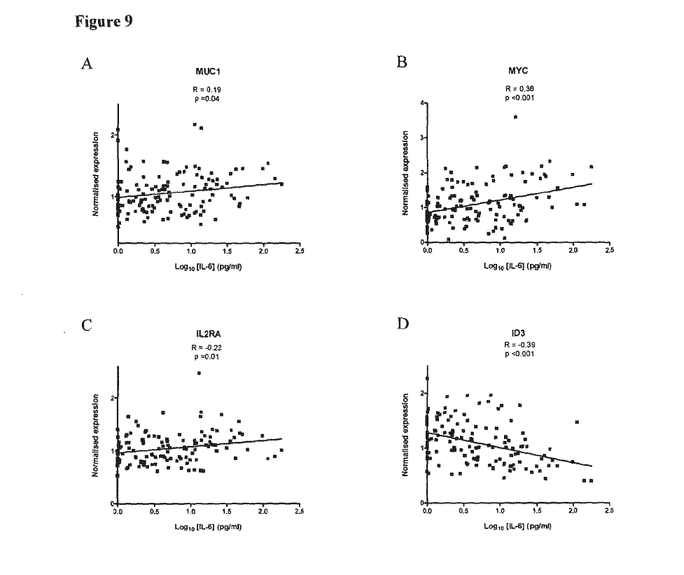

FIG. 4. (See FIG. 9 for additional examples). A-D. Serum IL-6 concentrations correlate with STAT3-inducible gene expression in PB CD4+ T-cells. Data are shown for 131 individuals in whom paired, contemporaneous samples were available; Pearson's R and associated p-values are shown.

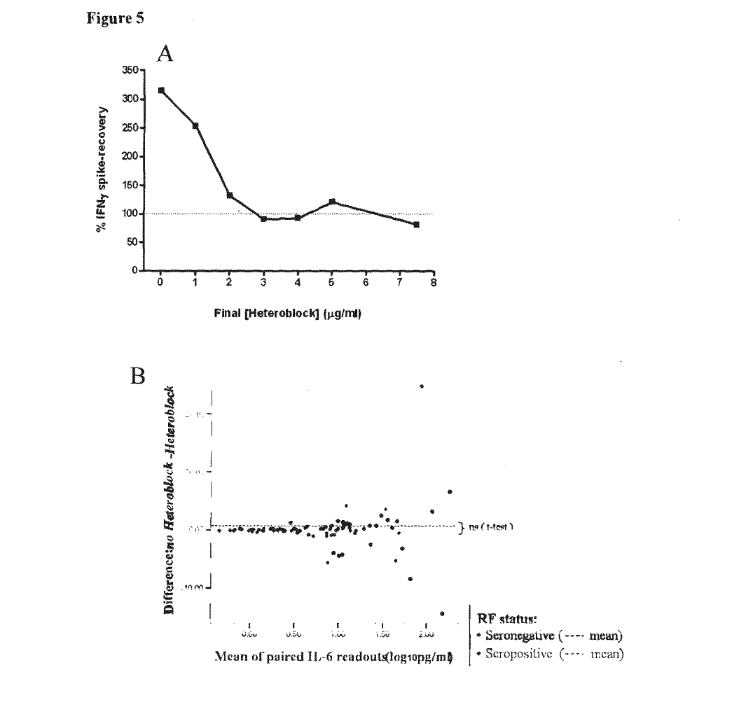

FIG. 5. A. Titration of proprietary cocktail of non-human sera (Heteroblock; see text) against IFN-γ spike recovery in exemplar RF+ human serum sample. In the absence of Heteroblock the difference in read-out between spiked and un-spiked samples (“spike recovery”) is significantly greater than the known spiked IFN-γ amount (>100%), indicating spuriously high assay readout due to the presence of heterophilic RF. Addition of ≧3 mg/ml final concentration of Heterblock neutralises this heterophilic effect. B. Bland-Altman plot of IL-6 readouts for 24 RF+ and 56 RF− serum samples obtained using MSD electrochemoluminescence platform, comparing assays performed in the presence/absence of a 3□g/ml final [Heteroblock]. No significant discrepancy is seen between RF+ and RF− samples in respect of the mean readout difference of the 2 assays. This indicates that the presence of potentially heterophilic antibodies is unlikely to affect assay readout in this system.

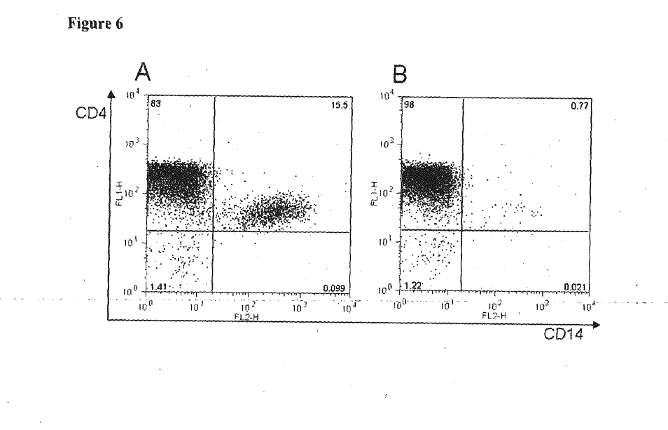

FIG. 6: Flow cytometric analysis of CD4+ positive-selection isolate before (A) and after (B) the monocyte-depletion step described in Methods. The extent of CD4+ CD14+ monocyte contamination varies, but may be as high as 15%, as in this example.

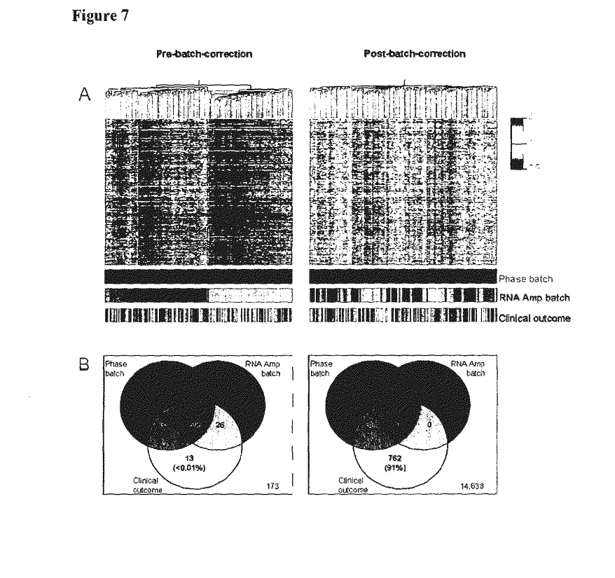

FIG. 7. Outputs for normalised expression data of 16,205 genes that passed filtering is shown amongst 173 samples before and after batch-correction using the method of Johnston et al (left and right panels respectively) (reference 23, amin text). A. Unsupervised hierarchical clustering of samples based on correlations in gene expression patterns (standard correlation, average linkage, represented by dendrogram). 173 samples are represented by columns and individual genes by rows; the colour at each co-ordinate indicates gene-wise fold-expression relative to median, according to the colour scale to the right of the figure. Underlying blue, red and yellow colour-bars label samples according to membership of phase batch (n=2), RNA amplification batch (n=6) and the clinical outcome category of interest (n=4; ACPA-negative RA, ACPA-positive RA, inflammatory or non-inflammatory controls). Artefactual clustering according to technical parameters (phase of study or within-phase RNA amplification batch) is eliminated through batch-correction, which does not of itself unmask clustering based on the clinical outcome of interest. B. Lists of genes that varied significantly (p<0.05 ANOVA) according to a sample's membership of phase batch (blue), RNA amplification batch (red) or clinical outcome of interest (yellow). Categories were generated amongst 16,205 passed genes, and overlapped in a Venn diagram. Without batch-correction virtually all genes seen to associate with clinical outcome are co-influenced by technical parameters. This potential source of technical bias is eliminated in 91% of outcome-related genes by the process of batch-correction. All genes named and discussed in this manuscript fell within this 91%.

FIG. 8. PB CD4+ T-cell expression profiles of indicated genes across 4 comparator groups, continued from FIG. 3; see Table 6 for characteristics of comparator groups. P-values shown are derived from non-parametric analysis of variance (Kruskall-Wallis); for post-hoc analyses, 1, 2 and 3 asterisks denote p<0.05, 0.01 and 0.001 respectively (Dunn's multiple comparison analysis).

FIG. 9. Serum IL-6 concentrations correlate with STAT3-inducible gene expression in PB CD4+ T-cells, continued from FIG. 4. Data are shown for 131 individuals in whom paired, contemporaneous samples were available; Pearson's R and associated p-values are shown.

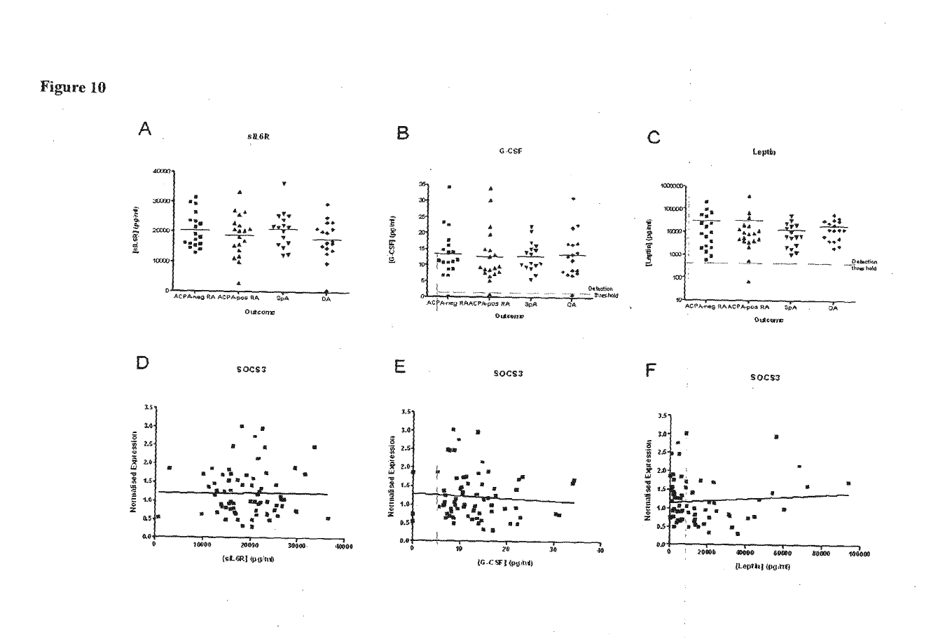

FIG. 10. A-C. No relationship between indicated serum analytes and diagnostic outcome amongst 80 early arthritis patients. Kruskall Wallis test; p>0.1 in all cases. D-F. Indicated serum analyte concentrations do not correlate with STAT3 gene expression (exemplar SOCS3 shown). Spearman's rank correlation; p>0.1 in all cases; and

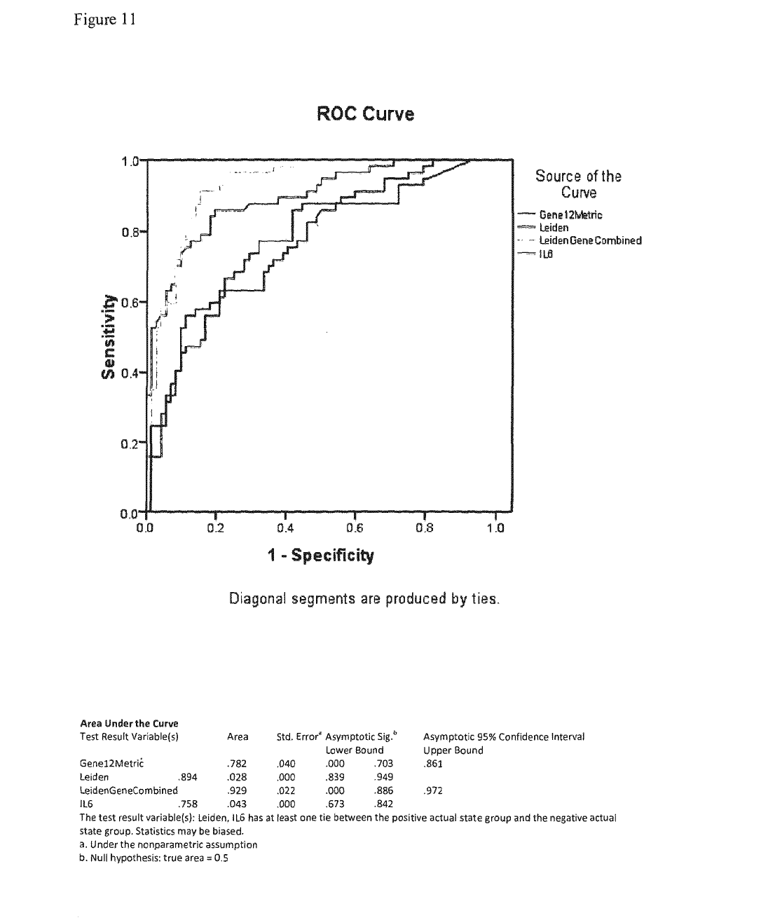

FIG. 11. A ROC curve for the whole cohort, including ACPA pos individuals, regardless of whether or not a diagnosis could be assigned at inception. The cohort includes 131 patients (all EA clinic attendees, including those with defined outcomes at inception); both ACPA+ and ACPA-; and

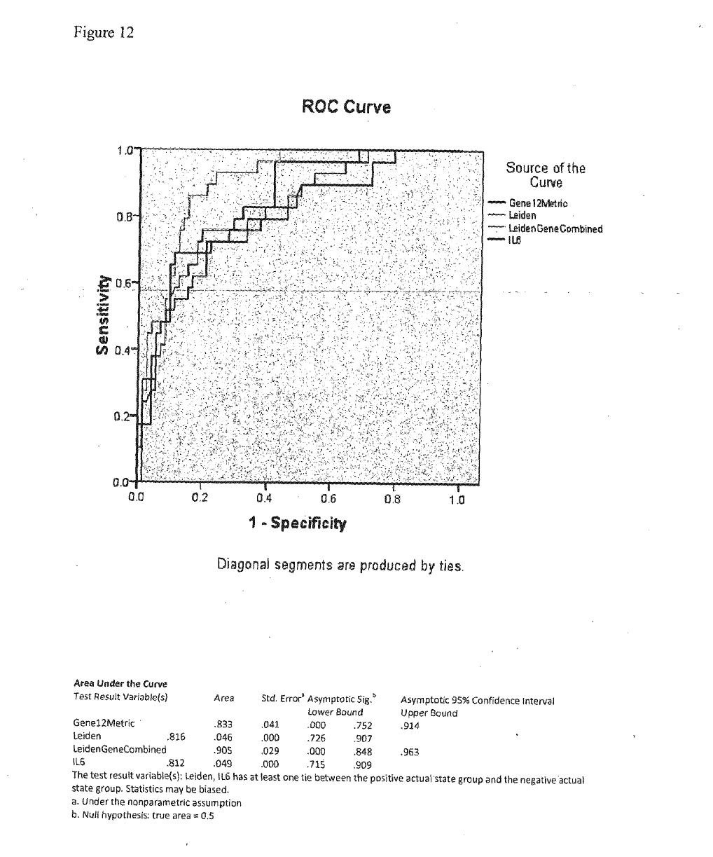

FIG. 12—A ROC curve for ACPA-neg individuals, but also regardless of whether or not a diagnosis could be assigned at inception. 102 patients (all ACPA-EA clinic attendees, including those with defined outcomes at inception). Amongst all ACPA-negative early arthritis clinic attendees, an [IL-6] of ≧10 pg/ml has approx. 0.89 specificity and 0.65 sensitivity for an outcome of RA; and

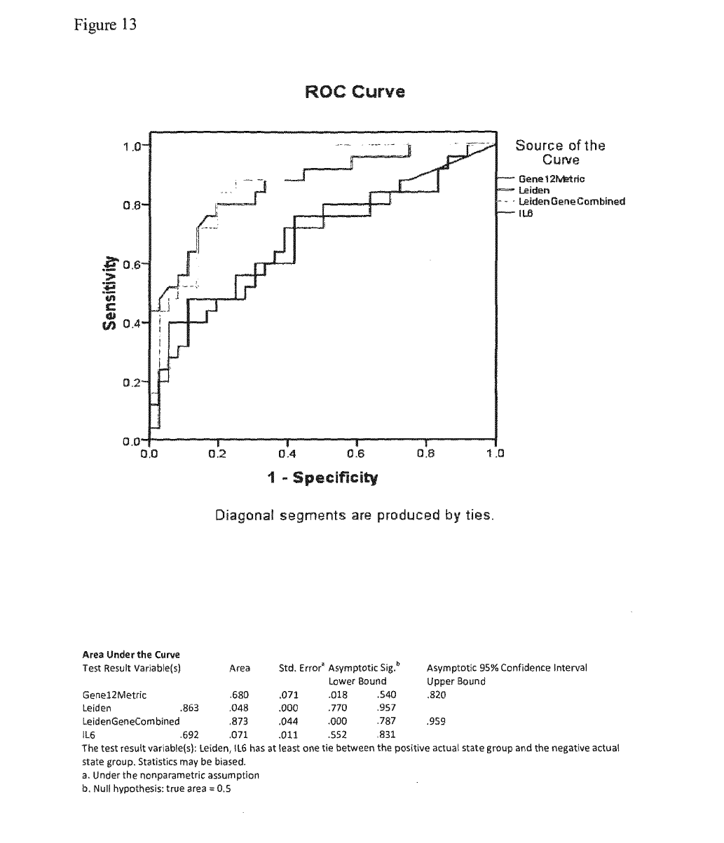

FIG. 13—A ROC curve for UA patients, whether they be ACPA-pos or ACPA-neg. 61 patients (UA patients only; both ACPA+ and ACPA-); and

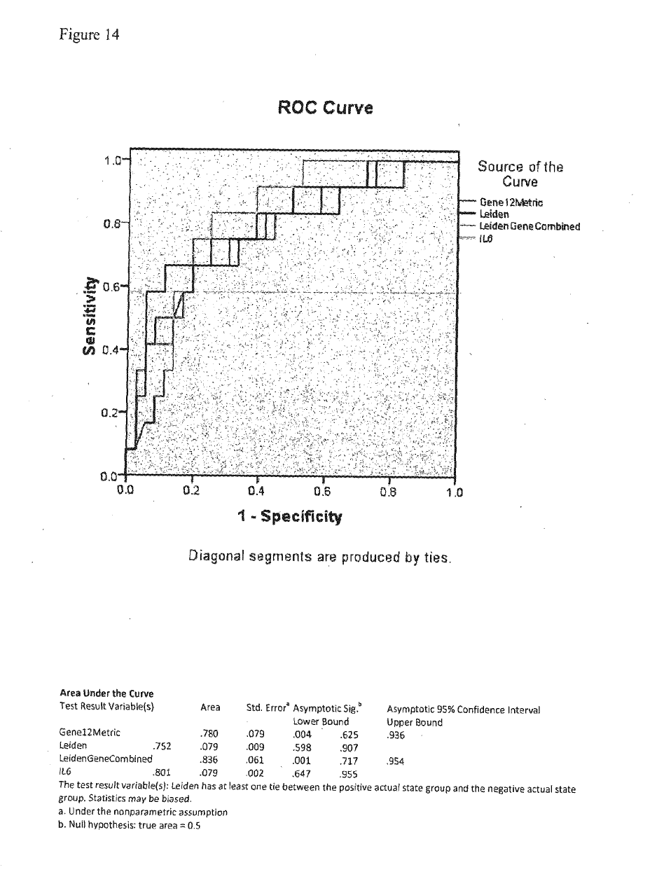

FIG. 14—A ROC curve for UA patients, ACPA-neg only. 48 patients (UA patients, ACPA-only). Amongst all ACPA-negative UA patients, an [IL-6] of ≧10 pg/ml has approx. 0.92 specificity and 0.58 sensitivity for an outcome of RA.

These examples are not to be considered as limiting.

### Patients and Methods

### Patients.

Patients with recent onset arthritis symptoms who were naïve to disease-modifying antirheumatic drugs (DMARDs) and corticosteroids, were recruited from the Freeman Hospital early arthritis clinic (EAC), Newcastle upon Tyne, UK, between September 2006 and December 2008. A detailed clinical assessment of each patient was undertaken, including ascertainment of ACPA status (anti-CCP2 test, Axis-Shield), along with routine baseline peripheral blood sampling. An initial working diagnosis was assigned to each patient according to a “working diagnosis proforma” (Table 3). RA was diagnosed only where 1987 ACR classification criteria(18) were unequivocally fulfilled, and UA was defined as a “suspected inflammatory arthritis where RA remained a possibility, but where established classification criteria for any rheumatological condition remained unmet”. This working diagnosis was updated by the consulting rheumatologist at each subsequent clinic visit for the duration of the study—a median of 28 months and greater than 12 months in all cases. The diagnostic outcome of patients with UA at inception was thereby ascertained, with individuals whose arthritis remained undifferentiated at the end of the study being excluded. Patients benefitted from routine clinical care for the duration of the investigation, and all gave written informed consent before inclusion into the study, which was approved by the Local Regional Ethics Committee.

TABLE 3
Categorisation of working diagnoses used amongst early arthritis patients
at inception and follow-up during the course of this study. Consultant
rheumatologists were asked to tick one box at each clinic visit, indicating
the best description of their expert opinion of the diagnosis at a given
time. See text.

| | | | |
|---|---|---|---|
| RA | | | □ |
| UA | | | □ |
| Non-RA: | “Inflammatory” | Psoriatic arthritis | □ |
| | | Reactive/self-limiting | □ |
| | | inflammatory arthritis | |
| | | Ankylosing spondylitis | □ |
| | | Enteropathic arthritis | □ |
| | | Undifferentiated spondyloarthritis | □ |
| | | (not RA) | |
| | | CTD | □ |
| | | Crystal | □ |
| | | Other | □ |
| | “Non-inflammatory” | Osteoarthritis | □ |
| | | Noninflammatory arthralgia/other. | □ |

### CD4+ T-Cell RNA Preparation.

Between 1300 hrs and 1630 hrs during the patients' EAC appointment, 15 ml peripheral whole blood was drawn into EDTA tubes (Greiner Bio-One, Austria) and stored at room temperature for a maximum of 4 hours before processing. Monocytes were first depleted by immunorosetting (Rosettesep® Human Monocyte depletion cocktail, Stemcell Technologies Inc., Vancouver, Canada), and remaining cells underwent positive selection using Easisep® whole blood CD4+ positive selection kit reagents in conjunction with the Robosep® automated cell separator (Stemcell). CD4+ T-cell purity was determined using standard flow cytometry techniques; FITC-conjugated anti-CD4 and PE-conjugated anti-CD14 antibodies were used (Beckton Dickinson, New Jersey, USA). RNA was immediately extracted from CD4+ T-cell isolates using RNeasy MINI Kits® (Qiagen GmbH, Germany), incorporating an “on-column” DNA digestion step.

### Microarrays.

Microarray experiments were performed in 2 phases (phase I, 95 samples; phase II, 78 samples). In each case, total RNA quality was assessed using an Agilent 2100 Bioanalyzer (Agilent Technologies, Palo Alto, Calif.) according to standard protocols(19). 250 ng RNA was reverse transcribed into cRNA, and biotin-UTP labeled, using the IIlumina TotalPrep RNA Amplification Kit (Ambion, Texas). cRNA was hybridised to the IIlumina Whole Genome 6 (version 3) BeadChip® (Illumina, San Diego, Calif.), following the manufacturer's protocol. Each BeadChip measured the expression of 48,804 genes (annotation file at http://www.illumina.com/support/annotation_files.ilmn) and was imaged using a BeadArray Reader (IIlumina).

### Serum Cytokine Measurement.

During baseline clinical assessment, blood was drawn into serum/gel tubes (Greiner Bio-One, Austria), and serum separated and frozen at −80° C. until use. Serum IL-6, sIL6R, TNF-a, leptin and G-CSF concentrations were measured using an immunosorbance assay platform that incorporates a highly sensitive electro-chemoluminescence detection system (Meso Scale Discovery [MSD], Gaithersberg, Md.) according to the manufacturer's instructions. The potential for heterophilic rheumatoid factors (RFs) in sera to cross-link capture and detection antibodies and contribute to spurious read-outs (20, 21) was excluded during pilot work (pilot Methods; FIG. 5).

qRT-PCR.

CD4+ T-cell total RNA samples were reverse transcribed using Superscript II® reverse transcriptase and random hexamers according to the manufacturer's instructions (Invitrogen, Carlsbad, Calif.). For replication of microarray findings real-time PCR reactions for reported transcripts were performed as part of a custom-made TaqMan Low Density Array (7900HT real-time PCR system, Applied Biosystems, Foster City, Calif.). Raw data were normalized and expressed relative to the housekeeping gene beta-actin (BACT) as 2^{-□Ct} values(22). BACT was selected from a panel of 9 potential housekeeping genes, having demonstrated optimal stability for this purpose.

### General Bioinformatics and Statistical Analysis.

Raw microarray data were imported into GeneSpring GX 7.3.1 software (Agilent Technologies), with which all statistical analyses were performed except where indicated. Phases I and II of the study were independently normalised in 2 steps: each probe measurement was first divided by the 50^{th} percentile of all measurements in its array, before being centred around its own median expression measurement across all samples in the phase. The anticipated batch-effect noted between phases on their combination, in addition to minor within-phase batch effects relating to one of the Illumina TotalPrep RNA Amplification steps, was corrected in the R statistical computing environment (http://www.r-project.org/) using the empirical Bayes method of Johnson et al(23). Raw and transformed data are available for review purposes at the Gene Expression Omnibus (GEO) address: http://www.ncbi.nlm.nih.gov/geo/query/acc.cgi?token=bviftkociimgsnk&acc=G SE20098. Genes detectably expressed (detection p-value <0.01(24)) in ≧1 sample of each study phase passed filtering of the normalised and batch-corrected data, and were included in subsequent analyses (16,205 genes). To define differential expression in this study. an arbitrary fold-change cut-off of 1.2 between comparator groups was combined with a significance level cut-off of p<0.05 (Welch's t-test), corrected for multiple testing using the false-discovery-rate (FDR) method of Benjamini et al(25). Genes identified in this way were used to train a support vector machine (SVM) classification model (Gaussian kernel) based on known outcomes amongst a “training” sample set(26). The model's accuracy, sensitivity and specificity as a prediction tool was then assessed amongst an independent “validation” sample set. In order to obtain larger lists of differentially expressed genes for biological pathway analysis, significance thresholds were subsequently relaxed through the omission of multiple-test-correction. Ingenuity Pathways Analysis software (Ingenuity Systems, Redwood City, Calif.) was used for the majority of these analyses. An objectively derived list of STAT3-inducible gene set was created for additional hypergeometric statistical testing by combining lists from two publically available databases (full list given in Gene List 1; sources; http://www.broadinstitute.org/gsea/msigdb/geneset_page.jsp?geneSetName=V$STAT3_{—}02&keywords=stat3 http://www.broadinstitute.org/gsea/msigdb/geneset_page.jsp?geneSetName=V$STAT3_{—}01&keywords=stat3 http://rulai.cshl.edu/cgi-bin/TRED/tred.cgi?process=searchTFGene&sel_type=factor_name&factor_organism=any&tx_search_terms=STAT3&target_organism=human&prom_quality=1&prom_quality=2&prom_quality=3&prom_quality=4&prom_quality=5&bind_quality=0&submit=SEARCH). Hypergeometric testing in this case was performed using Stat Trek on-line resource (http://stattrek.com). Parametric and non-parametric analyses of variance (ANOVAs), Mann-Whitney U tests, Pearson's correlation coefficients, intraclass correlations, multivariate analyses and the construction of receiver operator characteristic (ROC) curves were performed using SPSS version 15 (SPSS inc., Chicago Ill.).

### Derivation of Risk Metrics for ACPA-Negative UA.

Leiden prediction scores were calculated for each member of the training cohort according to baseline clinical and laboratory data as described in reference (5). Risk metrics based on the 12-gene RA “signature” were the sum of normalised expression values for the genes therein, assigning negative charge to the value for NOG (which was down-regulated in RA). Within the training dataset, both scores were entered as independent continuous variables into a logistic regression analysis with RA versus non-RA outcomes as the dependent variable (Table 4). In the resultant model the probability of an outcome of RA is related to both variables via the modified metric: B₁x₁+B₂x₂, where B₁ and B₂ are the regression coefficients for the Leiden prediction score and 12-gene risk metric respectively (B values in Table 4), and x₁ and x₂ are the values for each amongst individual patients. Hence, for a given patient the modified metric is equal to: (0.98×[Leiden prediction score])+(0.36×[12-gene risk metric/signature]).

TABLE 4
Results of logistic regression analysis for RA versus non-RA diagnoses
amongst 111 EA patients in the training cohort. B: regression coefficients;
SE(B): standard error for B; OR: odds ratio; CI: confidence interval.
The 12-gene risk metric/signature for a given patient is the sum of
normalised expression values for 12 genes in the putative RA signature
(value for NOG subtracted; see text). The Leiden prediction rule is
calculated according to reference (5). Both scores have independent
predictive value in discriminating clinical outcomes of interest. Regression
coefficients for each are used for the calculation of modified risk metrics
amongst the independent cohort of ACPA-negative UA patients (see text).

| Variable | B | SE (B) | Wald | p-value | OR (95% CI) |
|---|---|---|---|---|---|
| 12 gene risk | 0.36 | 0.1 | 11.0 | 0.001 | 1.4 (1.2-1.8) |
| metric/signature | | | | | |
| Leiden prediction rule | 0.98 | 0.2 | 21.4 | <0.001 | 2.5 (1.7-3.7) |
| Constant | −10.2 | 1.8 | 30.8 | <0.001 | — |

### Pilot Study Methods.

The potential for heterophilic rheumatoid factors (RFs) in sera to cross-link capture and detection antibodies and contribute to spurious read-outs was investigated in pilot work to this study. We first confirmed that a commercially available, proprietary cocktail of non-human sera (Heteroblock, Omega Biologicals Inc., Boseman, Mont.) could successfully neutralise the demonstrable heterophilic activity of native RF in human serum. A known final concentration of recombinant interferon-gamma (IFN-γ) was “spiked” into the sample and, by comparing the calculated difference in standard sandwich ELISA readout (BD Pharmingen, New Jersey, USA) between spiked and un-spiked samples with the actual spiked IFN-γ concentration, the extent of heterophilic activity could be ascertained, and the neutralising effect of varying concentrations of Heteroblock determined (FIG. 5A)(reference 21, main text). We next measured IL-6 concentration in 24 RF+ serum samples (median RF by nephelometry=165 IU) and 56 RE-negative samples, using the MSD platform, in each case running parallel assays with and without an optimised final concentration of Heteroblock. For the RF+ samples, excellent correlation was seen between assays performed with and without heteroblock (intraclass correlation coefficient=0.98 [95% CI=0.95-0.99]). A Bland-Altman plot confirmed that any such discrepancy that did exist was no less evident in RF-negative samples, suggesting that interference by heterophilic RFs in sera analysed using this platform is inconsequential (FIG. 5B). All serum measurements reported in the current study were therefore carried out using the MSD platform in the absence of Heteroblock.

### Results

### Patient Groups.

173 patient samples were retrospectively selected for microarray analysis. 111 of these originated from patients who could be assigned definitive diagnoses at inception, which were confirmed at a median follow-up of 28 months (minimum 1 year); an RA versus non-RA discriminatory “signature” was derived from this “training cohort” alone. The remaining 62 samples, all representing UA patients, formed an independent “validation cohort” for testing the utility of the “signature” according to diagnostic outcomes as they evolved during the same follow-up period. As expected, the characteristics of the UA cohort in respect of age, acute phase response, joint counts etc. fell between the equivalent measurements in the RA and control sample sets within the training cohort (Table 1). For subsequent pathway analysis, all 173 samples were pooled before being divided into four categories based on diagnostic outcome at the end of the study (Table 5).

TABLE 1
Clinical characteristics of the RA and Control comparator groups
used to generate list of differentially-expressed genes, which together
comprise a training cohort for machine-learning (total n = 111), and the
independent UA validation cohort (n = 62). Values are mean (1 SD range),
median (IQR) or % for normally-distributed, skewed or dichotomous data
respectively. ^{A} Statistical tests for significant difference between RA and Non-
RA groups; t-test, Mann-Whitney U or Fisher's exact test for normally-
distributed, skewed or dichotomous data respectively. Seroneg. spond:
seronegative sponyloarthropathy; CRP: C-reactive protein; RF: rheumatoid
factor; DAS28: disease activity score (incorporating 28-swollen/tender joint counts).

| | Training cohort | | | Test cohort |
|---|---|---|---|---|
| | RA | Non-RA | | UA |
| | (n = 47) | (n = 64) | p^{A} | (n = 62) |
| Age (years; mean, SD range) | 60 (46-74) | 48 (34-62) | <0.01 | 52 (37-67) |
| % Female | 65 | 61 | NS | 77 |
| % White Caucasian | 96 | 92 | NS | 90 |
| Symptom duration (weeks; median, IQR) | 12 (8-24) | 21 (10.5-52) | 0.026 | 14 (12-26) |
| Tender joint count (median, IQR) | 10 (4-15) | 7 (2-14) | 0.246 | 8 (3-16.5) |
| Swollen joint count (median, IQR) | 6 (2-10) | 0 (0-2) | <0.001 | 1 (0-3) |
| Morning stiffness (hours; median, IQR) | 1 (0.75-2) | 0.75 (0.1-2) | 0.007 | 1 (0.5-2) |
| ESR (s; median, IQR) | 56 (30-78) | 24 (14-52) | <0.001 | 30 (18-60) |
| CRP (g/l; median, IQR) | 17 (9-62) | 5 (2.5-19) | <0.001 | 8.5 (0-17) |
| % ACPA+ | 69 | 0 | — | 21 |
| % RF+ | 77 | 6 | — | 32 |
| DAS28 (median, IQR) | 5.37 | n/a | — | |
| Leiden prediction score (median, IQR) | n/a | n/a | | 6.4 (5-7.6) |
| Outcome Diagnosis (%) | | | | |
| RA | 100 | 0 | — | 40 |
| Seroneg Spond | — | 34 | — | 13 |
| Self-limiting inflam. | — | 19 | — | 15 |
| Other inflam. | — | 5 | — | 3 |
| OA/non-inflam. | — | 42 | — | 29 |

TABLE 5
Clinical characteristics of subjects as used in pathway analysis of pooled sample-set (n =
173), divided into 4 comparator groups by outcome at >1 year follow-up: ACPA-negative RA,
ACPA-positive RA, inflammatory and non-inflammatory control groups. Values are mean (1 SD
range), median (IQR) or % for normally-distributed, skewed or dichotomous data respectively.

| | ACPA- | ACPA- | Non-RA | Non-RA (OA/ | | |
|---|---|---|---|---|---|---|
| | neg RA | pos RA | Inflam^{y}. | non-inflam^{y}.) | p^{A} | p^{B} |
| | (n = 31) | (n = 41) | (n = 56) | (n = 45) | (3xInflam^{y}.) | (4xgroups) |
| Age | 61 | 56 | 44 | 52 | | |
| (years; mean, SD) | (46-77) | (44-70) | (30-60) | (40-64) | <0001 | <0.0001 |
| % Female | 66 | 61 | 62 | 80 | NS | NS |
| Symptom durn. | 12 | 12 | 12 | 32 | | |
| (wk; median, IQR) | (10-20) | (9-22) | (8-25) | (20-89) | NS | <0.0001 |
| Tender joint count | 10.5 | 10 | 5 | 9 | | |
| (median, IQR) | (5-15.5) | (3.5-16.5) | (2-13) | (2.5-19) | NS | NS |
| Swollen joint count | 4 | 3 | 1 | 0 | | |
| (median, IQR) | (1-4) | (0.5-7.5) | (0-4) | (0-0.5) | <0.001 | <0.0001 |
| Morning stiffness | 1 | 1 | 1 | 0.5 | | |
| (hrs; median, IQR) | (1-3.6) | (0.6-2.5) | (0.25-2) | (0.2-1.6) | NS | 0.005 |
| ESR | 48 | 54 | 34 | 20 | | |
| (s; median, IQR) | (27-68) | (27-73) | (20-72) | (9.5-30) | NS | <0.0001 |
| CRP | 18 | 10 | 13 | 2.5 | | |
| (g/l; median, IQR) | (10-57) | (5-35) | (5-23) | (2.5-6) | NS | <0.0001 |
| % ACPA+ | 0 | 100 | 0 | 0 | — | — |
| % RF+ | 25 | 93 | 11 | 12 | — | — |
| DAS28 | 4.9 | 5.2 | | | | |
| (median, IQR) | (4.5-5.9) | (4.1-6.0) | — | — | — | — |

^{A}statistical tests for significant variance between 3 inflammatory comparator groups (ACPA-negative RA, ACPA-positive RA and non-RA inflammatory arthritis); ANOVA, Krukskall-Wallis or Chi-square test normally-distributed, skewed or dichotomous data respectively.
^{B}statistical tests for significant variance between all 4 inflammatory comparator groups; ANOVA, Kruskall-Wallis or Chi square test for normally-distributed, skewed or dichotomous data respectively.

### CD4+ T-Cell Purity and Quality Control.

Flow cytometric analysis was completed for 148/173 (86%) of samples, and a median CD4+ CD14− purity of 98.9% was achieved (range 95-99.7%), with minimal CD4+ CD14+ monocyte contamination (median 0.32%; range 0.01-2.98%). Pilot work had demonstrated that incorporation of the monocyte depletion step described was required to achieve this (FIGS. 6A and B). RNA integrity numbers (RINs) for all 173 samples were calculated based on Agilent 2100 Bioanalyser(19), and all were of adequate quality for inclusion into microarray experiments (median RIN number 9.5). After normalisation of the raw data and filtering of expressed genes, technical bias relating to processing batches was successfully eliminated using the method of Johnson et al (FIG. 7)(23).

### RA Transcription “Signature” Most Accurate in ACPA-Negative UA.

Using a significance threshold robust to multiple test correction (false discovery rate p<0.05)(25), 12 non-redundant genes were shown to be differentially expressed (>1.2-fold) in PB CD4+ T-cells between 47 “training cohort” EAC patients with a confirmed diagnosis of RA, and 64 who could be assigned non-RA diagnoses (Table 2). An extended list, obtainable by omitting multiple-test correction, is given in Gene-List 2. Supervised hierarchical cluster analysis of the resultant multidimensional dataset (111 samples, 12 genes), demonstrated a clear tendency for EAC patients diagnosed with RA to cluster together based on this transcription profile (FIG. 1A). Quantitative real-time PCR (qRT-PCR) was used to analyse expression of seven of the differentially expressed genes in a subset of 73 samples. Despite the reduced power to detect change in this smaller dataset, robust differential expression was confirmed for six of the seven genes (Table 2).

TABLE 2
Fold-change and significance level for genes differentially
expressed at inception amongst PB CD4+ T-cells
between EAC patients with inception diagnoses of RA and
Non-RA (confirmed at ≧1 year; median 28 months
follow-up). The official gene symbol and RefSeq accession
number are given as identifiers, in boldface for 12 genes
included in statistically most robust “RA signature”,
and in regular text for additional STAT3-regulated genes
referred to in text.

| | | | | qRT-PCR | |
|---|---|---|---|---|---|
| | Microarray data | | | data | |
| | (47 RA vs. | | | (32 RA vs. | |
| | 64 non-RA) | | | 41 non-RA) | |
| Gene (Accn. No.) | \|FC\| | Uncorr. p^{A} | Corr. p^{A} | \|FC\| | p^{B} |
| 12-Gene RA Signature: | | | | | |
| BCL3 (NM_005178) | 1.59 | 2.6 × 10⁻⁵ | 0.03 | 2.15 | 0.005 |
| SOCS3 (NM_003955) | 1.55 | 3.4 × 10⁻⁶ | 0.03 | 1.83 | 0.002 |
| PIM1 (NM_002648) | 1.52 | 6.8 × 10⁻⁶ | 0.03 | 1.67 | 0.001 |
| SBNO2 (NM_014963) | 1.47 | 1.2 × 10⁻⁵ | 0.03 | 1.13 | 0.158 |
| LDHA (NM_005566) | 1.23 | 3.8 × 10⁻⁵ | 0.04 | 1.25 | 0.003 |
| CMAH (NR_002174) | 1.2 | 1.7 × 10⁻⁵ | 0.03 | 1.40 | 0.003 |
| NOG (NM_005450) | −1.32 | 3.1 × 10⁻⁵ | 0.03 | −1.59 | 0.004 |
| PDCD1 (NM_005018) | 1.42 | 1.0 × 10⁻⁵ | 0.03 | ND | ND |
| IGFL2 | 1.31 | 1.1 × 10⁻⁷ | 0.002 | ND | ND |
| (NM_001002915) | | | | | |
| LOC731186 | 1.28 | 2.3 × 10⁻⁵ | 0.03 | ND | ND |
| (XM_001128760) | | | | | |
| MUC1 | 1.26 | 2.0 × 10⁻⁵ | 0.03 | ND | ND |
| (NM_001044391) | | | | | |
| GPRIN3 (CR743148)^{C} | 1.32 | 2.1 × 10⁻⁴ | 0.049 | ND | ND |
| Additional | | | | | |
| STAT3-Regulated: | | | | | |
| ID3 (NM_002167) | −1.3 | 5.2 × 10⁻⁴ | 0.16 | ND | ND |
| MYC (NM_002467) | 1.2 | 0.04 | 0.75 | 1.29 | 0.01 |

ND: not done.
|FC|: linearised fold-change expression in RA relative to Non-RA (i.e. negative values represent genes down-regulated in RA relative to non-RA by n-fold).
^{A}Calculations based on normalised expression values of array data;
Welch's t-test, raw and multiple-test-corrected p-values given (see methods).
^{B}Calculations based on expression data normalised to

the house-keeping gene beta-actin (2^{-□Ct}); Mann-Whitney U test (see methods). ^{C}Note that the transcript CR743148 (IIlumina Probe ID 6370082) has been retired from NCBI, but the expressed sequence tag corresponds to splice variant(s) within the GPRIN3 gene (chromosome 4.90).

To derive a metric denoting risk of RA progression, the sum of normalised expression values for the 12-gene RA “signature” was calculated for each individual in the training cohort (see methods). A receiver operator characteristic (ROC) curve, plotting sensitivity versus [1-specificity] for a range of cut-offs of this risk metric, was then constructed, the area under which (0.85; standard error of mean [SEM]=0.04) suggested a promising discriminatory utility (FIG. 18). When the optimum discriminatory cut-off value for this metric based on the training cohort was applied to classify members of the validation cohort, RA could be predicted amongst UA patients with sensitivity, specificity, positive and negative likelihood ratios (95% CIs) accuracy of 0.64 (0.45-0.80), 0.70 (0.54-0.82), 2.2 (1.2-3.8) and 0.5(0.3-0.9) respectively. An alternative machine-learning methodology, a support vector machine (SVM), was also tested as a classification tool in our cohorts. Use of the SVM prediction model led to a modest improvement in UA classification accuracy over that of the ROC model, with sensitivity, specificity, positive and negative likelihood ratios (95% CIs) accuracy of 0.68 (0.48-0.83), 0.70 (0.60-0.87), 2.2 (1.2-3.8) and 0.4(0.2-0.8) respectively. However, we observed that of 13 ACPA-positive UA patients, 12 progressed to RA, indicating that autoantibody status alone was a much more sensitive predictor of RA in this subset. In contrast, when applied exclusively to the ACPA-negative subset of the UA validation cohort (n=49), the SVM classification model provided a sensitivity of 0.85 (0.58-0.96) and a specificity of 0.75 for progression to RA, thereby performing best in this diagnostically most challenging patient group. Hierarchical clustering of the ACPA-negative UA samples based on their 12-gene RA “signature” expression profiles further illustrates molecular similarities within the ACPA-negative RA outcome group (FIG. 1C).

The inventors have also found that a third gene could be included to make a 13 gene signature. Effectively, all genes are included as per the original 13 gene signature, but an additional down-regulated gene CD40LG is also included; this provides further specificity to the test giving an area under ROC curve of 0.835.

### Gene Signature Adds Value to Existing Tools in Diagnosing ACPA-Negative UA.

Next, we tested the potential additive diagnostic value of our 12-gene signature in comparison to the existing “Leiden prediction rule” as a predictor of RA amongst UA patients (4). Whilst the discriminatory utility achieved by the prediction rule in our UA cohort was comparable to that previously reported (n=62; AU ROC curve=0.86; SEM=0.05, data not shown), its performance diminished amongst the ACPA-negative sub-cohort (n=49; AU ROC curve=0.74; SEM=0.08; FIG. 1D. Employing a 12-gene risk metric as described above, equivalent discriminatory utility was found in this sub-cohort (AU ROC curve=0.78; SEM=0.08, data not shown). However, by deriving a modified risk metric, which combined all features of the Leiden prediction rule with our 12-gene risk signature (or 13 gene signature) (see Methods), and applying it to the independent ACPA-negative UA cohort, we could improve the utility of the prediction rule in this most diagnostically challenging patient group (AU ROC=0.84; SEM=0.06; FIG. 1D).

### A STAT3 Transcription Profile is Most Prominent in ACPA-Negative RA.

All 173 patients studied were now grouped into 4 categories based on outcome diagnosis alone: ACPA-positive RA, ACPA-negative RA, inflammatory non-RA controls and osteoarthritis (OA); their demographic and clinical characteristics are presented for comparison in Table 5. Three lists of differentially expressed genes could then be generated by comparing each of the “inflammatory” groups (which themselves exhibited comparable acute phase responses) with the OA group (>1.2 fold change; uncorrected p<0.05; Gene-lists 3-5). The 3 lists were overlapped on a Venn diagram (FIG. 2).

A highly significant over-representation of genes involved in the cell cycle was identified in association with ACPA-positive RA (24/46; p<1.0×10⁻⁵); FIG. 2; Gene-list 6). In addition, genes involved in the regulation of apoptosis were particularly over-represented in ACPA-negative RA patients, and RA was in general characterised by genes with functional roles in T-cell maturation (FIG. 2; gene-lists 6-9). Interestingly, within the highly significant 12-gene RA “signature” several genes (PIM1, SOCS3, SBNO2, BCL3 and MUC1) were noted to be STAT3-inducible based on literature sources (27-32). The majority of these were most markedly differentially expressed in ACPA-negative RA when compared to ACPA-positive RA (FIGS. 3A-B and FIGS. 8A-C). Additional STAT3-inducible genes (MYC, IL2RA; (27, 33, 34)) exhibited similar expression patterns, and there was a trend for STAT3 itself to be up-regulated in ACPA-negative RA compared to ACPA-positive RA (FIGS. 8D-F). Moreover, a reciprocal pattern of expression across outcome groups was observed for the dominant negative helix-loop-helix protein-encoding gene inhibitor of DNA-binding 3 (ID3) (FIG. 7G), consistent with its putative regulatory role in STAT3 signalling(35). MYC and ID3, although not included in the discriminatory RA signature under the stringent significance thresholds used, were nonetheless also seen to exhibit robust differential expression between RA and non-RA patients within the training cohort alone (Table 2). Finally, in relation to both the 12-gene signature, and the extended list of genes exclusively deregulated in ACPA-negative RA (Gene list 7), overlap with independently predicted STAT3-inducible gene sets (methods and Gene List 1) confirmed a preponderance of STAT3-inducible genes (hypergeometric p-values <0.005 in both cases)—which was not seen for genes deregulated only in ACPA-positive RA (p=0.19).

### Serum IL-6 is Highest in ACPA-Negative RA, and Independently Predicts CD4+ STAT3-Inducible Gene Expression.

Since one classical mechanism of STAT3 phosphorylation is via gp130 co-receptor ligation(36), we hypothesised that increased systemic levels of a key gp130 ligand and proinflammatory cytokine, IL-6, may be responsible for the STAT3-mediated transcriptional programme in early RA patients. Baseline serum IL-6 was measured in 131/173 EAC patients, subsequently grouped according to their ultimate diagnosis (ACPA-negative RA, ACPA-positive RA, non-RA inflammatory arthropathy or OA). IL-6 levels were low overall (generally <100 pg/ml), but were highest in the ACPA-negative RA group (FIG. 3C). Indeed, unlike the generic marker of systemic inflammation C-reactive protein (CRP), IL-6 had discriminatory value for ACPA-negative RA compared with non-RA inflammatory arthritides (FIGS. 3C and 3D). Furthermore, amongst individuals for whom paired and contemporaneous serum IL-6 and PB CD4+ T-cell RNA samples were obtained, a clear correlation was present between IL-6 and the normalised expression of a range of STAT3-inducible genes (FIGS. 4A-D; FIGS. 9A-D); for example, serum IL-6 measurements correlated with normalised SOCS3 expression: Pearson's R=0.57, p<0.001 (FIG. 4A). To exclude the possibility that this observation merely reflected systemic inflammation, multivariate analysis was carried out to measure the relative contribution of three related serum variables on STAT3-inducible gene expression. Hence, of CRP, IL-6 and the alternative pro-inflammatory cytokine tumour necrosis factor alpha (TNF-α, which does not signal via STAT3), only IL-6 independently predicted PB CD4+ T-cell SOCS3 expression amongst 131 early arthritis patients (β=0.53; p<0.001; Table 6). Finally, we found no similar relationship between alternative gp130 ligands measurable in sera (G-CSF and leptin) and PB CD4+ T-cell STAT3-inducible gene expression (FIG. 10).

TABLE 6
Results of standard linear regression analysis to identify related serum
variables independently associated with STAT-3 inducible gene
expression amongst 131 EA clinic patients. The dependent variable
was Log₁₀(normalised SOCS3 gene expression).

| | Unstandardised | | Standardised | | |
|---|---|---|---|---|---|
| | coefficients: | | coefficients: | | 95% CI (B) |
| Serum Variable | B | SE (B) | β | p-value | (lower, upper) |
| Log₁₀[IL-6] | 0.21 | 0.05 | 0.53 | <0.001 | 0.12, 0.30 |
| Log₁₀[CRP] | 0.06 | 0.04 | 0.13 | 0.18 | −0.03, 0.15 |
| Log₁₀[TNFα] | −0.09 | 0.09 | −0.08 | 0.32 | −0.27, 0.09 |
| Constant | −0.12 | 0.05 | — | 0.026 | −0.23, −0.02 |

SE (B): standard error for B;
CI: confidence interval. All variables underwent prior transformation in order to satisfy normality conditions of standard linear regression. Only serum [IL-6] is independently associated with CD4+ T-cell SOCS3 expression (p < 0.001; see text).

### Discussion.

We present a unique analysis of the ex-vivo PB CD4+ T-cell transcriptome in a well-characterised inception cohort of early arthritis patients. We have minimised confounding by including only patients naive to disease-modifying therapy, focussing on a single PB cell subset, collecting and processing samples expeditiously under standardised conditions, and employing careful quality control. In terms of a potential diagnostic tool, it is pleasing that our 12-gene “RA expression signature” (Table 2) performed best amongst the diagnostically challenging ACPA-negative UA patient group. These findings support the involvement of CD4+ T-cells in both ACPA positive and negative disease. The observation that both RA serotypes differed from a non-inflammatory control group to a greater extent than a non-RA inflammatory control group (FIG. 2), further supports this concept.

The signature's sensitivity and specificity (0.85 and 0.75) for predicting subsequent RA in seronegative UA patients equate to a positive likelihood ratio (LR+) of 3.4, indicating that a prior probability of 25% for RA progression amongst this cohort (13/49 patients progressed to RA) doubles to 53% for an individual assigned a positive SVM classification (posterior probability; [3.4×{0.25/0.75}]/[1+{3.4×(0.25/0.75)}](37)). Moreover, of the 13 ACPA-negative UA patients who progressed to RA in our cohort, 8 fell into an “intermediate” risk category for RA progression according to the validated Leiden prediction score(4), thereby remaining subject to delayed diagnosis. Encouragingly, all but one of these patients were correctly classified based on their 12-gene expression profiles. Our proof-of-concept that this approach might add value to existing algorithms in the diagnosis of ACPA-negative UA is further supported by the construction of ROC curves comparing the Leiden prediction rule with a modified risk metric that amalgamates the features of our gene signature with those of the prediction rule (FIG. 1D). Further validation of the RA signature in well-defined ACPA-negative UA populations is now a priority.

Our data indicate that PB CD4+ T-cells in early RA are characterised by a predominant up-regulation of biological pathways involved in cell cycle progression (ACPA-positive) and survival (ACPA-negative) (FIG. 2 and Gene-lists 6-7). Pathway analysis also suggested that T-cell development and differentiation were de-regulated in both RA serotypes (Gene-list 8). These findings are consistent with previous observations of impaired T-cell homeostasis in RA, characterised by increased turnover, telomere shortening and immunosenescence (38). Intriguingly, such observations may be associated with carriage of HLA-DRB1 shared epitope alleles(39), which have themselves since been defined as risk-factors for ACPA positivity (40), consistent with the more marked CD4+ T-cell-cycling programme in seropositive individuals suggested by our study (FIG. 2).

Given the well-characterised importance of the STAT3 signalling pathway in both oncogenesis and T-cell survival pathways, it was notable that 5 genes from our statistically robust 12-gene RA signature are reportedly induced following STAT3 phosphorylation(27-32). This up-regulation was generally most pronounced in ACPA-negative RA (FIGS. 3A-B and FIG. 8A-C), potentially explaining why the predictive utility of the 12-gene signature was optimal in this disease subset. Additional STAT3-inducible genes (including IL2RA and MYC(27, 33, 34)), along with STAT3 itself, exhibited similar, albeit less statistically robust, expression patterns across the clinical comparator groups, and a reciprocal pattern was seen for ID3 expression, consistent with a proposed regulatory function of its product with respect to STAT3 signalling (35) (FIG. 8D-G; see also Gene-list 7). Our observation that increased serum IL-6 levels amongst early arthritis clinic attendees may predict a diagnosis of RA versus alternative arthritides is consistent with findings of previous biomarker studies(41, 42), but ours is, to our knowledge, the first demonstration of a particular association with ACPA-negative disease (FIG. 3C).

Striking correlations were seen between PB CD4+ T-cell expression of several STAT3-inducible genes and paired, contemporaneous serum IL-6 concentrations (FIGS. 4 A-D; FIGS. 9A-D). Although IL-6 measurements also correlated with systemic inflammation in general (measured as CRP), as well as serum levels of an alternative, non-STAT3-signalling pro-inflammatory cytokine, TNF-α (data not shown), multivariate analysis confirmed IL-6 to be the sole independent predictor of STAT3 gene expression (Table 6). STAT3 phosphorylation and downstream transcription is initiated by ligation of the cell-surface gp130 co-receptor by a range of ligands including IL-6 (43). We measured IL-6 in particular because of its recognised role as a pro-inflammatory cytokine in RA(44). However, given that only 30-50% of PB CD4+ T-cells are thought to express membrane-bound IL6R(45), it was possible that the critical in vivo determinant of at least some STAT-3 inducible gene-expression in this setting might be circulating sIL6-R rather than IL-6 levels. Trans-signalling of IL-6 via the soluble form of the receptor is crucial for IL-6 mediated responses in IL-6R-negative T-cells. We therefore measured baseline Serum sIL-6R concentrations in a subset of 80 early arthritis patients from the current study, comprising 20 of each diagnostic outcome defined in Table 2. In contrast to IL-6, no relationship with diagnostic outcome was detected, and neither was there a correlation between serum sIL-6R concentration and STAT3-inducible gene expression (FIGS. 10A and D).

The inventors also studied two other gp130 ligands seeking a potential role for them in STAT3 pathway induction; Granulocyte colony stimulating factor (G-CSF) and leptin have both been implicated in RA pathogenesis(46, 47), but their levels in sera from the same subset of study patients neither correlated with diagnostic outcome nor STAT3 gene expression (FIGS. 10 B-C, E-F). Finally, IL-10, which is also known to signal through STAT3(48), was undetectable in the majority of sera (data not shown). It therefore seems likely that the findings in relation to a STAT3 inducible gene expression signature as part of an early arthritis biomarker for seronegative RA are largely specific to IL-6 signalling.

The inventors also reviewed ROC curves to look at the discriminatory utility of various scoring systems in their cohort, and subsets thereof. 42 patients were excluded from the analysis for whom no IL-6 measurements were available, however only one of these presented with UA.

FIGS. 11-14, look at:

FIG. 11—The whole cohort, including ACPA pos individuals, regardless of whether or not a diagnosis could be assigned at inception.
FIG. 12—ACPA-neg individuals, but also regardless of whether or not a diagnosis could be assigned at inception.
FIG. 13—UA patients, whether they be ACPA-pos or ACPA-neg
FIG. 14—UA patients, ACPA-neg only.

In each cohort/sub-cohort, 4 ROC curves are compared: the Leiden prediction rule, the 12-gene risk metric we discussed, the composite Leiden/12-gene metric mentioned in the manuscript, and IL-6 alone.

In slides 12 and 14 (excluding ACPA-positive patients), there are given sensitivities/specificities for an example cut-off of 10 pg/ml serum [IL-6].

The results suggest that IL-6 is a useful parameter for predicting outcome in early ACPA-negative disease in particular. The most effective prediction appears to be given by a composite of the Leiden prediction score and the 12-gene metric.

In conclusion, the data provides strong evidence for the induction of an IL-6-mediated STAT3 transcription programme in PB CD4+ T-cells of early RA patients, which is most prominent in ACPA-negative individuals, and which contributes to a gene expression “signature” that may have diagnostic utility. Such a pattern of gene expression amongst CD4+ T-cells at this critical early phase in the natural history of inflammatory arthritis could have a defining role in the switch from potentially self-limiting inflammation to T-cell-perpetuated chronic autoimmunity—a model which may not be limited to the example of RA. In any event, the findings could pave the way for a novel treatment paradigm in early arthritis, whereby drugs targeting the IL-6-gp130-STAT3 “axis” find a rational niche as first choice biologic agents in the management of ACPA-negative RA. One such agent, already available in the clinic, is the IL-6 receptor blocker tocilizumab, whose efficacy is already established in RA(49); others include janus kinase inhibitors currently undergoing phase III clinical trials for the disease (50). Studies such as ours should ultimately contribute to the realisation of true “personalised medicine” in early inflammatory arthritis, in which complex heterogeneity is stratified into pathophysiologically and therapeutically relevant subsets, with clear benefits in terms of clinical outcome and cost.

Gene List 1 - STAT3-INDUCIBLE GENES on ILLUMINA ARRAY

| | | Symbol (source |
|---|---|---|
| Symbol (Illumina) | RefSeq | database, if different) |
| A2M | NM_000014.4 | |
| ACCN1 | NM_001094.4 | |
| ACCN4 | NM_018674.3 | |
| ADM2 | NM_024866.4 | |
| ALKBH6 | NM_198867.1 | MGC14376 |
| AP1S2 | NM_003916.3 | |
| AP2B1 | NM_001282.2 | |
| APBA1 | NM_001163.2 | |
| ARF3 | NM_001659.1 | |
| ARHGAP8 | NM_181335.2 | |
| ARL6IP6 | NM_152522.3 | MGC33864 |
| ARX | NM_139058.1 | |
| ASXL1 | NM_015338.4 | |
| ATG3 | NM_022488.3 | |
| AZIN1 | NM_015878.4 | OAZIN |
| B3GAT3 | NM_012200.2 | |
| BCAM | NM_005581.3 | LU |
| BCL2 | NM_000633.2 | |
| BCL2L1 | NM_138578.1 | |
| BCL7A | NM_001024808.1 | |
| BIRC5 | NM_001168.2 | |
| BMI1 | NM_005180.5 | |
| BMP4 | NM_130851.1 | |
| BNC1 | NM_001717.2 | |
| BTBD1 | NM_001011885.1 | |
| C14orf179 | NM_052873.1 | MGC16028 |
| C16orf85 | NM_001001682.1 | FLJ45530 |
| C17orf91 | NM_001001870.1 | MGC14376 |
| C5orf41 | NM_153607.1 | LOC153222 |
| CA10 | NM_020178.3 | |
| CALU | NM_001219.2 | |
| CAPZA1 | NM_006135.1 | |
| CCL2 | NM_002982.3 | |
| CCND1 | NM_053056.2 | |
| CCND3 | NM_001760.2 | |
| CD40 | NM_152854.2 | TNFRSF5 |
| CDKN1A | NM_000389.2 | |
| CEBPB | NM_005194.2 | |
| CENTD1 | NM_139182.1 | |
| CHRM1 | NM_000738.2 | |
| CISH | NM_145071.1 | |
| CLDN5 | NM_003277.2 | |
| COL4A3BP | NM_005713.1 | |
| CPA4 | NM_016352.2 | |
| CPLX2 | NM_006650.3 | |
| CRTAC1 | NM_018058.4 | |
| CSRP1 | NM_004078.1 | |
| CTGF | NM_001901.2 | |
| CXorf36 | NM_024689.1 | FLJ14103 |
| CYP19A1 | NM_000103.2 | |
| DDIT3 | NM_004083.4 | |
| DERL2 | NM_016041.3 | F-LANA |
| EGR1 | NM_001964.2 | |
| EGR3 | NM_004430.2 | |
| EHHADH | NM_001966.2 | |
| EIF4E | NM_001968.2 | |
| EIF4G1 | NM_198244.1 | |
| EIF5A | NM_001970.3 | |
| ELMO1 | NM_130442.2 | |
| EPHA7 | NM_004440.2 | |
| EXOC3 | NM_007277.4 | SEC6L1 |
| FAS | NM_152872.1 | TNFRSF6 |
| FASN | NM_004104.4 | |
| FBN2 | NM_001999.3 | |
| FBXL3 | NM_012158.1 | FBXL3A |
| FCGR1A | NM_000566.2 | |
| FLJ33387 | NM_182526.1 | |
| FLRT1 | NM_013280.4 | |
| FOS | NM_005252.2 | |
| FOSB | NM_006732.1 | |
| FOXO4 | NM_005938.2 | MLLT7 |
| FUT8 | NM_178156.1 | |
| GABRB1 | NM_000812.2 | |
| GEN1 | NM_182625.2 | FLJ40869 |
| GPC3 | NM_004484.2 | |
| GPHN | NM_001024218.1 | |
| GRIN2D | NM_000836.2 | |
| HEYL | NM_014571.3 | |
| HMOX1 | NM_002133.1 | |
| HNRPR | NM_005826.2 | |
| HOXB13 | NM_006361.5 | |
| HOXB4 | NM_024015.3 | |
| HOXB9 | NM_024017.3 | |
| HOXC4 | NM_014620.4 | |
| HOXC6 | NM_153693.3 | |
| HS6ST3 | NM_153456.2 | |
| HSP90AA1 | NM_001017963.2 | HSPCA |
| HSP90AB1 | NM_007355.2 | HSPCB |
| ICAM1 | NM_000201.1 | |
| IGF1 | NM_000618.2 | |
| IL10 | NM_000572.2 | |
| IL18BP | NM_005699.2 | |
| IL2RA | NM_000417.1 | |
| IL6 | NM_000600.1 | |
| IL6ST | NM_002184.2 | |
| IRF1 | NM_002198.1 | |
| IRX5 | NM_005853.4 | |
| JAK3 | NM_000215.2 | |
| JUN | NM_002228.3 | |
| KAZALD1 | NM_030929.3 | |
| KCNH3 | NM_012284.1 | |
| KCNN2 | NM_021614.2 | |
| KCNN3 | NM_002249.4 | |
| KCNT2 | NM_198503.2 | SLICK |
| KIAA0146 | NM_001080394.1 | |
| KIAA0913 | NM_015037.2 | |
| KIRREL3 | NM_032531.2 | |
| KPNB1 | NM_002265.4 | |
| LBP | NM_004139.2 | |
| LRP2 | NM_004525.2 | |
| LTA | NM_000595.2 | |
| LTBP1 | NM_000627.2 | |
| MAFF | NM_012323.2 | |
| MAML1 | XM_937023.1 | |
| MATN4 | NM_030592.1 | |
| MBD6 | NM_052897.3 | |
| MCL1 | NM_021960.3 | |
| MEIS2 | NM_172315.1 | |
| MIA2 | NM_054024.3 | |
| MID1IP1 | NM_021242.4 | MIG12 |
| MIS12 | NM_024039.1 | |
| MLL | NM_005933.2 | |
| MNT | NM_020310.2 | |
| MOBKL2C | NM_201403.2 | |
| MTMR14 | NM_001077525.1 | FLJ22405 |
| MUC1 | NM_002456.4 | |
| MUC4 | NM_018406.3 | |
| MYC | NM_002467.3 | |
| MYT1 | NM_004535.2 | |
| NAPB | NM_022080.1 | |
| NAV2 | NM_145117.3 | |
| NCAM1 | NM_001076682.2 | |
| NCOA5 | NM_020967.2 | |
| NDST2 | NM_003635.2 | |
| NELL2 | NM_006159.1 | |
| NFAM1 | NM_145912.5 | |
| NOL3 | NM_003946.3 | |
| NOS2A | NM_000625.3 | |
| NPAS4 | NM_178864.2 | NXF |
| NR1D1 | NM_021724.2 | |
| NR4A1 | NM_002135.3 | |
| OSM | NM_020530.3 | |
| OXTR | NM_000916.3 | |
| PAPD1 | NM_018109.2 | |
| PCBP4 | NM_033009.1 | |
| PGF | NM_002632.4 | |
| PIM1 | NM_002648.2 | |
| PPFIA2 | NM_003625.2 | |
| PRF1 | NM_005041.4 | |
| PROS1 | NM_000313.1 | |
| PTMS | NM_002824.4 | |
| RBPJ | NM_203283.1 | PRBPSUH |
| RBPJL | NM_014276.2 | RBPSUHL |
| REG1A | NM_002909.3 | |
| REM2 | NM_173527.2 | FLJ38964 |
| RGS3 | NM_134427.1 | |
| RIMS1 | NM_014989.3 | |
| RND1 | NM_014470.2 | |
| RNF213 | NM_020914.3 | C17ORF27 |
| RORA | NM_002943.2 | |
| RPUSD4 | NM_032795.1 | FLJ14494 |
| RSPO4 | NM_001040007.1 | R-SPONDIN |
| S100A14 | NM_020672.1 | |
| SCUBE3 | NM_152753.2 | |
| SDC1 | NM_002997.4 | |
| SENP3 | NM_015670.4 | |
| SERPING1 | NM_001032295.1 | |
| SET | NM_003011.2 | |
| SGMS1 | NM_147156.3 | TMEM23 |
| SHOX2 | NM_006884.2 | |
| SLC35A5 | NM_017945.2 | |
| SLC38A5 | NM_033518.1 | |
| SLCO5A1 | NM_030958.1 | |
| SMG7 | NM_201569.1 | C1ORF16 |
| SOCS1 | NM_003745.1 | |
| SOCS3 | NM_003955.3 | |
| SOS1 | NM_005633.2 | |
| SP6 | NM_199262.2 | |
| SPON1 | NM_006108.2 | |
| SPTBN2 | NM_006946.1 | APG3 |
| ST7L | NM_138729.2 | |
| STRA13 | NM_144998.2 | |
| SV2B | NM_014848.3 | |
| TAOK1 | NM_020791.1 | TAO1 |
| TCF7L2 | NM_030756.2 | |
| TIMP1 | NM_003254.2 | |
| TIMP3 | NM_000362.4 | |
| TJAP1 | NM_080604.1 | TJP4 |
| TLR2 | NM_003264.3 | |
| TM9SF1 | NM_006405.5 | |
| TMEM158 | NM_015444.2 | |
| TMEM180 | NM_024789.3 | C10ORF77 |
| TMEM37 | NM_183240.2 | PR1 |
| TNF | NM_000594.2 | |
| TNFRSF8 | NM_001243.3 | |
| TNFSF18 | NM_005092.2 | |
| TNRC6A | NM_014494.2 | TNRC6 |
| TRAF4 | NM_004295.3 | |
| TRH | NM_007117.1 | |
| TRIB2 | NM_021643.1 | |
| TRIP10 | NM_004240.2 | |
| TSC22D4 | NM_030935.3 | THG-1 |
| UBE4B | NM_006048.2 | |
| UBR1 | NM_174916.1 | |
| UBR5 | NM_015902.4 | DD5 |
| UBTF | NM_014233.2 | |
| UPK2 | NM_006760.2 | |
| VCL | NM_014000.2 | |
| VEGFA | NM_003376.4 | VEGF |
| VEZF1 | NM_007146.2 | ZNF161 |
| VIP | NM_194435.1 | |
| VSNL1 | NM_003385.4 | |
| WDR81 | NM_152348.1 | FLJ33817 |
| WEE1 | NM_003390.2 | |
| WNT4 | NM_030761.3 | |
| YY1 | NM_003403.3 | |
| ZBTB11 | NM_014415.2 | |
| ZBTB17 | NM_003443.1 | ZNF151 |
| ZBTB25 | NM_006977.2 | ZNF46 |
| ZBTB9 | NM_152735.3 | |
| ZC3H18 | NM_144604.2 | LOC124245 |
| ZFP112 | NM_001083335.1 | ZNF228 |
| ZFYVE9 | NM_007324.2 | |
| ZHX2 | NM_014943.3 | |
| ZNF296 | NM_145288.1 | ZNF342 |
| ZNF395 | NM_018660.2 | PBF |

Note, for hypergeometric probabilities, total number of “non-redundant” genes used as “population size” = 37,847

Gene-List 2: RA vs Non-RA, Training samples (n = 111)

| | | | | un- |
|---|---|---|---|---|
| | | | | corrected |
| Illumina ID | Symbol | RefSeq | FC | p* |
| 2070168 | CX3CR1 | NM_001337.3 | 1.657 | 0.0308 |
| [symbol] | [symbol] | [symbol] | [symbol] | [symbol] |
| [symbol] | [symbol] | [symbol] | [symbol] | [symbol] |
| 430438 | MIAT | NR_003491.1 | 1.531 | 0.000239 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| 6220288 | PRDM1 | NM_001198.2 | 1.415 | 0.000242 |
| 60470 | STX11 | NM_003764.2 | 1.386 | 0.00117 |
| 6620689 | MTHFD2 | NM_001040409.1 | 1.375 | 7.88E−05 |
| 1510553 | DACT1 | NM_016651.5 | 1.37 | 0.00763 |
| 4670193 | PRF1 | NM_005041.4 | 1.351 | 0.0465 |
| 1710070 | ITGAM | NM_000632.3 | 1.339 | 0.0249 |
| 5700753 | CEACAM1 | NM_001024912.1 | 1.334 | 0.000235 |
| 160292 | APOBEC3H | NM_181773.2 | 1.332 | 0.0017 |
| 6220195 | BATF | NM_006399.2 | 1.326 | 0.000953 |
| [symbol] | [symbol] | [symbol] | [symbol] | [symbol] |
| 7200301 | ARID5A | NM_212481.1 | 1.321 | 0.00388 |
| [symbol] | [symbol] | [symbol] | [symbol] | [symbol] |
| 4060358 | ABCA1 | NM_005502.2 | 1.299 | 0.00162 |
| 6550600 | MYC | NM_002467.3 | 1.295 | 0.0425 |
| 6770673 | SOCS2 | NM_003877.3 | 1.287 | 0.011 |
| [symbol] | [symbol] | [symbol] | [symbol] | [symbol] |
| 650452 | PLCH2 | NM_014638.2 | 1.275 | 0.00242 |
| 7560731 | SNORA64 | NR_002326.1 | 1.265 | 0.000326 |
| 1430598 | FBXO32 | NM_058229.2 | 1.264 | 0.00127 |
| 2070037 | ICOS | NM_012092.2 | 1.263 | 0.00318 |
| 5490068 | MCOLN2 | NM_153259.2 | 1.26 | 0.0131 |
| 3800647 | UGCG | NM_003358.1 | 1.258 | 0.00242 |
| 4230228 | CDK5RAP3 | NM_176095.1 | 1.257 | 0.0126 |
| [symbol] | [symbol] | [symbol] | [symbol] | [symbol] |
| 2070288 | MT1E | NM_175617.3 | 1.253 | 0.0171 |
| 1410408 | ARID5B | NM_032199.1 | 1.248 | 0.000716 |
| 1510424 | S100P | NM_005980.2 | 1.246 | 0.000653 |
| 3420128 | AP3M2 | NM_006803.2 | 1.246 | 0.00305 |
| 3190148 | DDIT4 | NM_019058.2 | 1.245 | 0.0281 |
| 160494 | AQP9 | NM_020980.2 | 1.243 | 0.0275 |
| 870202 | TNFSF10 | NM_003810.2 | 1.24 | 0.00949 |
| 2320129 | CSDA | NM_003651.3 | 1.235 | 0.0405 |
| 2100215 | MAF | NM_001031804.1 | 1.233 | 0.00222 |
| 6420731 | SLC20A1 | NM_005415.3 | 1.231 | 9.77E−05 |
| 10333 | LOC731682 | XM_001129369.1 | 1.229 | 0.0356 |
| 4540376 | FAM13A1 | NM_001015045.1 | 1.228 | 0.043 |
| 1850554 | NPDC1 | NM_015392.2 | 1.227 | 0.000194 |
| [symbol] | [symbol] | [symbol] | [symbol] | [symbol] |
| 4260372 | GTSCR1 | XM_496277.2 | 1.225 | 0.049 |
| 6250010 | GPRIN3 | NM_198281.2 | 1.223 | 0.0136 |
| 3840470 | ST6GALNAC1 | NM_018414.2 | 1.222 | 0.0141 |
| 4590446 | MSL3L1 | NM_078628.1 | 1.22 | 0.00221 |
| 5890524 | LINS1 | NM_181740.1 | 1.22 | 0.000796 |
| 7570600 | FLJ33590 | NM_173821.1 | 1.22 | 0.00361 |
| 3940390 | TBXAS1 | NM_001061.2 | 1.218 | 0.0186 |
| 450615 | MT2A | NM_005953.2 | 1.216 | 0.000379 |
| 520278 | FAM100B | NM_182565.2 | 1.214 | 0.00367 |
| 7050326 | CDKN2D | NM_079421.2 | 1.214 | 0.000125 |
| 1400601 | C20orf100 | NM_032883.1 | 1.212 | 0.0142 |
| 5130382 | CLDN5 | NM_003277.2 | 1.212 | 0.0321 |
| 5700735 | PARP9 | NM_031458.1 | 1.211 | 0.00126 |
| 5910364 | TYMS | NM_001071.1 | 1.211 | 0.00678 |
| 5900471 | PTGER2 | NM_000956.2 | 1.21 | 0.0115 |
| 2850291 | GARNL4 | NM_015085.3 | 1.209 | 0.00831 |
| 4180301 | ZNF365 | NM_014951.2 | 1.209 | 0.0192 |
| 4200541 | FAM113B | NM_138371.1 | 1.209 | 0.000487 |
| 5090754 | KIAA0101 | NM_014736.4 | 1.208 | 0.00994 |
| 7100372 | PRPF4B | NM_003913.3 | 1.206 | 0.0112 |
| 5420538 | TP53INP1 | NM_033285.2 | 1.205 | 0.016 |
| 1510364 | GBP5 | NM_052942.2 | 1.204 | 0.0182 |
| 3800168 | SLC2A3 | NM_006931.1 | 1.204 | 0.0331 |
| 3840053 | UGP2 | NM_006759.3 | 1.204 | 0.000214 |
| 4610201 | SNORA10 | NR_002327.1 | 1.203 | 8.50E−05 |
| 6200168 | PIM2 | NM_006875.2 | 1.203 | 0.000135 |
| 2190689 | OSBPL5 | NM_020896.2 | 1.202 | 0.0208 |
| 2760112 | P2RY5 | NM_005767.4 | 1.201 | 0.00774 |
| 4670603 | ELMO2 | NM_133171.2 | 1.201 | 0.0101 |
| 3460008 | TMEM173 | NM_198282.1 | 1.2 | 0.000813 |
| 3780161 | TMEM70 | NM_017866.4 | 1.2 | 0.00548 |
| [symbol] | [symbol] | [symbol] | [symbol] | [symbol] |
| 3170703 | LY9 | NM_001033667.1 | 0.837 | 0.0043 |
| 5810746 | MATN2 | NM_002380.3 | 0.837 | 0.00284 |
| 5080615 | IL16 | NM_172217.2 | 0.835 | 0.0262 |
| 160242 | C13orf15 | NM_014059.2 | 0.834 | 0.00866 |
| 6200019 | KLRB1 | NM_002258.2 | 0.831 | 0.0393 |
| 6280504 | LOC100008589 | NR_003287.1 | 0.83 | 0.0364 |
| 6280243 | DNTT | NM_001017520.1 | 0.829 | 0.000589 |
| 5130692 | DDX17 | NM_030881.3 | 0.821 | 0.0105 |
| 2970730 | MYADM | NM_001020820.1 | 0.819 | 0.0171 |
| 870056 | FAM119B | NM_015433.2 | 0.816 | 0.00801 |
| 1580477 | C11orf74 | NM_138787.2 | 0.815 | 7.68E−05 |
| 2710309 | ELA1 | NM_001971.4 | 0.783 | 0.00177 |
| 50706 | CD40LG | NM_000074.2 | 0.78 | 0.000441 |
| 1470762 | AUTS2 | NM_015570.1 | 0.779 | 0.0224 |
| 7570324 | ID3 | NM_002167.2 | 0.775 | 0.000522 |
| 2320253 | USMG5 | NM_032747.2 | 0.774 | 0.0236 |
| [symbol] | [symbol] | [symbol] | +z, | [symbol] |
| 130609 | FCGBP | NM_003890.1 | 0.707 | 0.00124 |
| 5080192 | SERPINE2 | NM_006216.2 | 0.657 | 0.00192 |

*Red/Bold/Italicised entries => p < 0.05 when corrected for multiple-testing (FDR)
**transcript CR743148 (Illumina Probe ID 6370082) has been retired from NCBI, but the EST corresponds to splice variant(s) within the GPRIN3 gene (chromosome 4.90).

Gene-List 3: ACPA-neg RA vs OA, Pooled dataset (n = 173)

| Illumina | | | | uncorrected |
|---|---|---|---|---|
| ID | Symbol | RefSeq | FC | p* |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| 510079 | HLA-DRB4 | NM_021983.4 | 1.701 | 0.0231 |
| [symbol] | [symbol] | [symbol] | [symbol] | [symbol] |
| 1820594 | HBEGF | NM_001945.1 | 1.607 | 0.00284 |
| [symbol] | [symbol] | [symbol] | [symbol] | [symbol] |
| [symbol] | [symbol] | [symbol] | [symbol] | [symbol] |
| 6290270 | MNDA | NM_002432.1 | 1.558 | 0.0499 |
| 670010 | LOC650298 | XM_939387.1 | 1.555 | 0.033 |
| 60470 | STX11 | NM_003764.2 | 1.553 | 0.000765 |
| 6220288 | PRDM1 | NM_001198.2 | 1.531 | 0.000238 |
| 6550600 | MYC | NM_002467.3 | 1.527 | 0.00645 |
| 6590377 | RPS26 | NM_001029.3 | 1.527 | 0.0216 |
| [symbol] | [symbol] | [symbol] | [symbol] | [symbol] |
| 4230201 | CDKN1A | NM_000389.2 | 1.505 | 0.0136 |
| 1240152 | CFD | NM_001928.2 | 1.499 | 0.0314 |
| 4670048 | RPS26L | NR_002225.2 | 1.499 | 0.0372 |
| 1990300 | SOCS1 | NM_003745.1 | 1.49 | 0.0155 |
| 6270307 | LOC644934 | XM_930344.2 | 1.476 | 0.0265 |
| 6370082 | GPRIN3** | CR743148 | 1.457 | 8.53E−05 |
| 4060358 | ABCA1 | NM_005502.2 | 1.454 | 0.000771 |
| 7200301 | ARID5A | NM_212481.1 | 1.448 | 0.00212 |
| [symbol] | [symbol] | [symbol] | [symbol] | [symbol] |
| 6770673 | SOCS2 | NM_003877.3 | 1.441 | 0.00609 |
| 2070037 | ICOS | NM_012092.2 | 1.438 | 0.000297 |
| [symbol] | [symbol] | [symbol] | [symbol] | [symbol] |
| 430438 | MIAT | NR_003491.1 | 1.428 | 0.00396 |
| 6560376 | RPS26L1 | NR_002309.1 | 1.423 | 0.0367 |
| 6250010 | GPRIN3 | NM_198281.2 | 1.419 | 0.00082 |
| 1440736 | LDLR | NM_000527.2 | 1.415 | 0.00256 |
| 870202 | TNFSF10 | NM_003810.2 | 1.404 | 0.000859 |
| 3710397 | EFNA1 | NM_004428.2 | 1.402 | 0.000713 |
| 2650192 | C6orf105 | NM_032744.1 | 1.399 | 0.000885 |
| 5870692 | GPR132 | NM_013345.2 | 1.399 | 0.0278 |
| 50672 | GSTM1 | NM_000561.2 | 1.394 | 0.0462 |
| 1510553 | DACT1 | NM_016651.5 | 1.374 | 0.0373 |
| 670255 | GADD45A | NM_001924.2 | 1.373 | 0.0411 |
| 2320129 | CSDA | NM_003651.3 | 1.369 | 0.0161 |
| 3940438 | NCF1 | NM_000265.4 | 1.369 | 0.0486 |
| 6960195 | LOC650646 | XM_942527.2 | 1.369 | 0.0456 |
| 6280458 | BCL6 | NM_001706.2 | 1.363 | 0.00517 |
| 520278 | FAM100B | NM_182565.2 | 1.352 | 0.000191 |
| 160494 | AQP9 | NM_020980.2 | 1.349 | 0.0216 |
| 7400747 | FAM89A | NM_198552.1 | 1.336 | 0.000375 |
| 6860347 | FAM46C | NM_017709.3 | 1.334 | 0.0429 |
| 1430598 | FBXO32 | NM_058229.2 | 1.331 | 0.0017 |
| 2570291 | IFNGR2 | NM_005534.2 | 1.329 | 0.000131 |
| 3800168 | SLC2A3 | NM_006931.1 | 1.329 | 0.00763 |
| 3840470 | ST6GALN | NM_018414.2 | 1.329 | 0.00551 |
| | AC1 | | | |
| 4670603 | ELMO2 | NM_133171.2 | 1.322 | 0.00404 |
| 270152 | SLC7A5 | NM_003486.5 | 1.321 | 0.0382 |
| 5700753 | CEACAM1 | NM_001024912.1 | 1.321 | 0.00359 |
| [symbol] | [symbol] | [symbol] | [symbol] | [symbol] |
| 4810520 | TRIB1 | NM_025195.2 | 1.316 | 0.0366 |
| 5670465 | ADM | NM_001124.1 | 1.313 | 0.0187 |
| [symbol] | [symbol] | [symbol] | [symbol] | [symbol] |
| 4730411 | SFXN1 | NM_022754.4 | 1.312 | 0.00143 |
| 5270097 | LOC653853 | XM_936029.1 | 1.309 | 0.00641 |
| 1510424 | S100P | NM_005980.2 | 1.305 | 0.00177 |
| [symbol] | [symbol] | [symbol] | [symbol] | [symbol] |
| 5890524 | LINS1 | NM_181740.1 | 1.298 | 0.000974 |
| 3420128 | AP3M2 | NM_006803.2 | 1.296 | 0.00528 |
| 1030102 | RGS16 | NM_002928.2 | 1.295 | 0.000337 |
| 3190148 | DDIT4 | NM_019058.2 | 1.291 | 0.0213 |
| 3460008 | TMEM173 | NM_198282.1 | 1.287 | 0.000248 |
| 6280672 | TMEM49 | NM_030938.2 | 1.284 | 0.000283 |
| 5820020 | PRDX3 | NM_006793.2 | 1.282 | 0.0019 |
| 2470348 | NFKBIZ | NM_001005474.1 | 1.281 | 0.00621 |
| 2470358 | IFNGR1 | NM_000416.1 | 1.279 | 0.00205 |
| 7560731 | SNORA64 | NR_002326.1 | 1.278 | 0.00259 |
| 1230201 | CTLA4 | NM_005214.3 | 1.277 | 0.00417 |
| 450348 | GNG10 | NM_001017998.2 | 1.276 | 9.60E−05 |
| 380056 | B3GNT2 | NM_006577.5 | 1.274 | 0.000643 |
| 1990753 | SLA | NM_006748.1 | 1.271 | 0.0131 |
| 2600735 | TLR6 | NM_006068.2 | 1.271 | 0.00765 |
| 3840053 | UGP2 | NM_006759.3 | 1.271 | 0.000201 |
| 2070288 | MT1E | NM_175617.3 | 1.27 | 0.0397 |
| 6270554 | LGALS8 | NM_201545.1 | 1.27 | 0.000789 |
| 2640341 | FKBP5 | NM_004117.2 | 1.269 | 0.00778 |
| 6660630 | TP53INP1 | NM_033285.2 | 1.266 | 0.00444 |
| 4590446 | MSL3L1 | NM_078628.1 | 1.265 | 0.00248 |
| 4610201 | SNORA10 | NR_002327.1 | 1.265 | 0.00016 |
| 4010097 | FBXO5 | NM_012177.2 | 1.264 | 0.000131 |
| 1260086 | ID2 | NM_002166.4 | 1.263 | 0.014 |
| 7320041 | GALNAC4S- | NM_015892.2 | 1.262 | 0.0289 |
| | 6ST | | | |
| 4670414 | TMEM140 | NM_018295.2 | 1.261 | 0.00276 |
| 3870706 | FURIN | NM_002569.2 | 1.259 | 0.00518 |
| 1410408 | ARID5B | NM_032199.1 | 1.258 | 0.00417 |
| 4200541 | FAM113B | NM_138371.1 | 1.258 | 0.000767 |
| 4590228 | GLRX | NM_002064.1 | 1.258 | 0.00719 |
| 20446 | CEBPB | NM_005194.2 | 1.257 | 0.0108 |
| 1850554 | NPDC1 | NM_015392.2 | 1.257 | 0.00132 |
| 5550343 | PDCL | NM_005388.3 | 1.253 | 0.000603 |
| 840554 | RYBP | NM_012234.4 | 1.251 | 0.00531 |
| 1340075 | BAG3 | NM_004281.3 | 1.251 | 0.000391 |
| 4230554 | REXO2 | NM_015523.2 | 1.251 | 0.00173 |
| 5420564 | NFIL3 | NM_005384.2 | 1.251 | 0.0156 |
| 6220543 | HIF1A | NM_001530.2 | 1.251 | 0.0068 |
| 70167 | LY96 | NM_015364.2 | 1.249 | 0.00105 |
| 4230619 | GCA | NM_012198.2 | 1.249 | 0.0104 |
| 2760112 | P2RY5 | NM_005767.4 | 1.247 | 0.0114 |
| 6420731 | SLC20A1 | NM_005415.3 | 1.247 | 0.000324 |
| [symbol] | [symbol] | [symbol] | [symbol] | [symbol] |
| 3420593 | LMNB1 | NM_005573.2 | 1.245 | 0.00222 |
| 4590349 | ACVR2A | NM_001616.3 | 1.245 | 0.0009 |
| 630167 | SDCBP | NM_001007067.1 | 1.244 | 0.0149 |
| 2750719 | DDX21 | NM_004728.2 | 1.243 | 0.00255 |
| 5130382 | CLDN5 | NM_003277.2 | 1.241 | 0.0235 |
| 1010653 | POLR1C | NM_203290.1 | 1.239 | 0.00639 |
| 10630 | IL21R | NM_181078.1 | 1.237 | 0.00733 |
| 5270167 | GNL3 | NM_206826.1 | 1.237 | 0.00236 |
| 6200168 | PIM2 | NM_006875.2 | 1.237 | 0.000751 |
| 3190112 | SERPINB1 | NM_030666.2 | 1.236 | 0.000149 |
| 6280170 | PDCD1 | NM_005018.1 | 1.236 | 0.0285 |
| 670086 | MXD1 | NM_002357.2 | 1.235 | 0.0026 |
| 2230379 | NAMPT | NM_005746.2 | 1.232 | 0.0132 |
| 3890326 | SOD2 | NM_001024465.1 | 1.232 | 0.0147 |
| 4050681 | NDUFV2 | NM_021074.1 | 1.232 | 0.00154 |
| 6370414 | CLECL1 | NM_172004.2 | 1.232 | 0.0459 |
| 2060615 | ACVR1B | NM_020328.2 | 1.23 | 0.000616 |
| 2710709 | FCGR1B | NM_001017986.1 | 1.229 | 0.0434 |
| 5270110 | EIF4A3 | NM_014740.2 | 1.228 | 0.000397 |
| 4920110 | GADD45B | NM_015675.2 | 1.227 | 0.00136 |
| 6380112 | GRAMD4 | NM_015124.2 | 1.227 | 0.000299 |
| 2190452 | PIM3 | XM_938171.2 | 1.225 | 0.00047 |
| 5900274 | EDA | NM_001005611.1 | 1.225 | 0.000448 |
| 2630400 | CSTF2T | NM_015235.2 | 1.224 | 0.00143 |
| 7150176 | MAT2A | NM_005911.4 | 1.224 | 0.00517 |
| 5080021 | BIRC3 | NM_001165.3 | 1.22 | 0.00923 |
| 4260019 | NGRN | NM_016645.2 | 1.218 | 0.0008 |
| 4810615 | SLC25A44 | NM_014655.1 | 1.218 | 0.00342 |
| 5870307 | LOC440359 | XM_496143.2 | 1.218 | 0.0223 |
| 1990630 | TRIB3 | NM_021158.3 | 1.217 | 0.0221 |
| 2600059 | GNPDA1 | NM_005471.3 | 1.215 | 0.0146 |
| 5900471 | PTGER2 | NM_000956.2 | 1.213 | 0.0365 |
| 3930390 | SMAP2 | NM_022733.1 | 1.212 | 0.00839 |
| 3420241 | SLC2A14 | NM_153449.2 | 1.211 | 0.0374 |
| 5360079 | GIMAP5 | NM_018384.3 | 1.211 | 0.00847 |
| 130452 | GP5 | NM_004488.1 | 1.21 | 0.000553 |
| 5810504 | METRNL | NM_001004431.1 | 1.21 | 0.0219 |
| 4120131 | KISS1R | NM_032551.3 | 1.209 | 0.00165 |
| 1030646 | FLJ43692 | NM_001003702.1 | 1.208 | 0.00793 |
| 4480504 | ZNF828 | NM_032436.1 | 1.207 | 0.00585 |
| 270601 | HIAT1 | NM_033055.2 | 1.206 | 0.0116 |
| 990735 | RNF149 | NM_173647.2 | 1.205 | 0.0033 |
| 3170091 | GIMAP7 | NM_153236.3 | 1.203 | 0.00101 |
| 3390612 | TLR8 | NM_016610.2 | 1.203 | 0.0404 |
| 1940524 | STS-1 | NM_032873.3 | 1.202 | 0.00732 |
| 4900575 | PTRH2 | NM_016077.3 | 1.202 | 0.0103 |
| 130021 | IL2RA | NM_000417.1 | 1.2 | 0.00378 |
| 2100484 | STAT3 | NM_139276.2 | 1.2 | 0.00317 |
| 60079 | DNAJB1 | NM_006145.1 | 0.831 | 0.00833 |
| 3170703 | LY9 | NM_001033667.1 | 0.83 | 0.0178 |
| 7610440 | XAF1 | NM_199139.1 | 0.827 | 0.0389 |
| 4200475 | MAST3 | NM_015016.1 | 0.824 | 0.0359 |
| 770161 | C10orf73 | XM_096317.11 | 0.813 | 0.00616 |
| 870056 | FAM119B | NM_015433.2 | 0.811 | 0.0225 |
| 3610300 | CCDC58 | NM_001017928.2 | 0.811 | 0.0149 |
| 5080615 | IL16 | NM_172217.2 | 0.806 | 0.0306 |
| 3990170 | IFI27 | NM_005532.3 | 0.799 | 0.0267 |
| 6770603 | NOG | NM_005450.2 | 0.779 | 0.000954 |
| 7570324 | ID3 | NM_002167.2 | 0.75 | 0.00109 |
| 50706 | CD40LG | NM_000074.2 | 0.739 | 0.000205 |
| 2230538 | LRRN3 | NM_001099660.1 | 0.578 | 0.0308 |

*Red/Bold/italicised entries => p < 0.05 when corrected for multiple-testing (FDR)
**transcript CR743148 (Illumina Probe ID 6370082) has been retired from NCBI, but the EST corresponds to splice variant(s) within the GPRIN3 gene (chromosome 4.90).

Gene-List 4: ACPA-pos RA vs OA, Pooled dataset (n = 173)

| Illumina | | | | |
|---|---|---|---|---|
| ID | Symbol | RefSeq | FC | uncorrected p* |
| 5080692 | HLA-A29.1 | NM_001080840.1 | 1.961 | 0.0149 |
| 430438 | MIAT | NR_003491.1 | 1.546 | 0.000406 |
| 3130301 | PIM1 | NM_002648.2 | 1.536 | 0.000107 |
| 6220288 | PRDM1 | NM_001198.2 | 1.485 | 0.000136 |
| 4230102 | SOCS3 | NM_003955.3 | 1.452 | 0.000601 |
| 6330725 | BCL3 | NM_005178.2 | 1.434 | 0.00186 |
| 6280170 | PDCD1 | NM_005018.1 | 1.427 | 1.73E−05 |
| 1400601 | C20orf100 | NM_032883.1 | 1.381 | 0.000272 |
| [symbol] | [symbol] | [symbol] | [symbol] | [symbol] |
| 6220195 | BATF | NM_006399.2 | 1.372 | 0.00029 |
| 2070037 | ICOS | NM_012092.2 | 1.37 | 7.70E−05 |
| 3190609 | SBNO2 | NM_014963.2 | 1.369 | 0.000182 |
| 5090754 | KIAA0101 | NM_014736.4 | 1.337 | 0.000717 |
| 5910364 | TYMS | NM_001071.1 | 1.334 | 0.00037 |
| 6620689 | MTHFD2 | NM_001040409.1 | 1.332 | 0.000143 |
| 6370082 | | CR743148 | 1.331 | 6.90E−05 |
| 1990300 | SOCS1 | NM_003745.1 | 1.328 | 0.0283 |
| 1230201 | CTLA4 | NM_005214.3 | 1.326 | 0.000122 |
| 60470 | STX11 | NM_003764.2 | 1.311 | 0.00495 |
| 2070520 | CDCA7 | NM_031942.4 | 1.311 | 0.00086 |
| 3800647 | UGCG | NM_003358.1 | 1.308 | 0.000353 |
| 130022 | CDCA5 | NM_080668.2 | 1.305 | 0.000177 |
| 7560731 | SNORA64 | NR_002326.1 | 1.3 | 6.93E−05 |
| 5420538 | TP53INP1 | NM_033285.2 | 1.29 | 0.00336 |
| 6250010 | GPRIN3 | NM_198281.2 | 1.288 | 0.00228 |
| 2570253 | BTN3A2 | NM_007047.3 | 1.285 | 0.017 |
| 4060358 | ABCA1 | NM_00 [symbol] 502.2 | 1.262 | 0. [symbol] 22 |
| 4260368 | UBE2C | NM_181800.1 | 1.278 | 0.00038 |
| 10333 | LOC731682 | XM_001129369.1 | 1.274 | 0.00834 |
| 160292 | APOBEC3H | NM_181773.2 | 1.274 | 0.0101 |
| 4230228 | CDK5RAP3 | NM_176095.1 | 1.273 | 0.0141 |
| 5420095 | MYC | NM_002467.3 | 1.273 | 0.00207 |
| [symbol] | [symbol] | [symbol] | [symbol] | [symbol] |
| 1850554 | NPDC1 | NM_015392.2 | 1.272 | 0.000954 |
| 3990619 | TOP2A | NM_001067.2 | 1.269 | 0.000693 |
| 5360070 | CCNB2 | NM_004701.2 | 1.258 | 0.000841 |
| 3840470 | ST6GAL- | NM_018414.2 | 1.256 | 0.00534 |
| | NAC1 | | | |
| 3780161 | TMEM70 | NM_017866.4 | 1.255 | 0.000225 |
| 520278 | FAM100B | NM_182565.2 | 1.252 | 0.00317 |
| 1430598 | FBXO32 | NM_058229.2 | 1.246 | 0.00115 |
| 5890524 | LINS1 | NM_181740.1 | 1.246 | 0.000377 |
| 3610440 | MAF | NM_005360.3 | 1.245 | 0.00787 |
| 6350189 | MGC4677 | NM_052871.3 | 1.239 | 0.0214 |
| 1500010 | CDC20 | NM_001255.2 | 1.236 | 0.000944 |
| 2320170 | CDC45L | NM_003504.3 | 1.236 | 4.66E−05 |
| 5700753 | CEACAM1 | NM_001024912.1 | 1.236 | 0.0226 |
| 5340246 | CRIP2 | NM_001312.2 | 1.235 | 0.0179 |
| 1410408 | ARID5B | NM_032199.1 | 1.234 | 0.00269 |
| 1690692 | SOCS2 | NM_003877.3 | 1.233 | 0.0309 |
| 2470348 | NFKBIZ | NM_001005474.1 | 1.232 | 0.00421 |
| 4880646 | FKSG30 | NM_001017421.1 | 1.232 | 0.0191 |
| 1450056 | CPA5 | NM_080385.3 | 1.231 | 0.00564 |
| 1500553 | NUSAP1 | NM_018454.5 | 1.23 | 0.00215 |
| 1710019 | ICA1 | NM_004968.2 | 1.23 | 0.0016 |
| 4730411 | SFXN1 | NM_022754.4 | 1.225 | 0.000836 |
| 4810520 | TRIB1 | NM_025195.2 | 1.224 | 0.0324 |
| 4890750 | DDX11 | NM_030653.3 | 1.224 | 0.0376 |
| 2490161 | CLEC2B | NM_005127.2 | 1.222 | 0.0378 |
| 10414 | PTTG1 | NM_004219.2 | 1.219 | 0.00173 |
| 1510364 | GBP5 | NM_052942.2 | 1.218 | 0.0161 |
| 380056 | B3GNT2 | NM_006577.5 | 1.216 | 0.000916 |
| 2940110 | UHRF1 | NM_001048201.1 | 1.216 | 0.00165 |
| 3190092 | LDHA | NM_005566.1 | 1.215 | 9.71E−05 |
| 3420241 | SLC2A14 | NM_153449.2 | 1.21 | 0.00873 |
| 6100408 | NLRC5 | NM_032206.3 | 1.21 | 0.00475 |
| 7570600 | FLJ33590 | NM_173821.1 | 1.21 | 0.012 |
| 7650026 | MUC1 | NM_001044391.1 | 1.21 | 0.0017 |
| 450615 | MT2A | NM_005953.2 | 1.209 | 0.00164 |
| 1450280 | NCAPG | NM_022346.3 | 1.208 | 0.000123 |
| 160097 | MELK | NM_014791.2 | 1.207 | 2.95E−05 |
| 4730196 | TK1 | NM_003258.2 | 1.207 | 0.000283 |
| 5090528 | CYorf15B | NM_032576.2 | 1.207 | 0.0415 |
| 6480053 | ATF4 | NM_001675.2 | 1.206 | 0.0166 |
| 4610189 | HERPUD1 | NM_001010990.1 | 1.205 | 0.0217 |
| 4830056 | ARPC5L | NM_030978.1 | 1.204 | 6.82E−05 |
| 3800168 | SLC2A3 | NM_006931.1 | 1.203 | 0.0313 |
| 5260600 | ZNF655 | NM_001009957.1 | 1.203 | 0.00963 |
| 7200301 | ARID5A | NM_212481.1 | 1.202 | 0.0349 |
| 2600735 | TLR6 | NM_006068.2 | 1.201 | 0.0157 |
| 2760112 | P2RY5 | NM_005767.4 | 1.201 | 0.0133 |
| 2970730 | MYADM | NM_001020820.1 | 0.818 | 0.0113 |
| 3610300 | CCDC58 | NM_001017928.2 | 0.815 | 0.0182 |
| 50706 | CD40LG | NM_000074.2 | 0.81 | 0.00637 |
| 6770603 | NOG | NM_005450.2 | 0.802 | 0.00116 |
| 7570324 | ID3 | NM_002167.2 | 0.757 | 8.06E−05 |
| 130609 | FCGBP | NM_003890.1 | 0.688 | 0.00193 |
| 2230538 | LRRN3 | NM_001099660.1 | 0.679 | 0.0483 |
| 5080192 | SERPINE2 | NM_006216.2 | 0.628 | 0.0069 |
| 7050021 | PRKAR1A | NM_002734.3 | 0.522 | 0.00561 |

*Red/Bold/italicised entries => p < 0.05 when corrected for multiple-testing (FDR) [symbol]
indicates data missing or illegible when filed

Gene-List 5: Non-RA inflam. vs OA, Pooled dataset (n = 173)

| Illumina | | | | |
|---|---|---|---|---|
| ID | Symbol | RefSeq | FC | uncorrected p* |
| 3610743 | SF1 | NM_201997.1 | 1.658 | 0.0427 |
| 6960661 | FAM118A | NM_017911.1 | 1.595 | 0.0349 |
| 1430113 | LOC728505 | XM_001127580.1 | 1.366 | 0.000994 |
| 1690440 | XIST | NR_001564.1 | 1.358 | 0.013 |
| 6560376 | RPS26L1 | NR_002309.1 | 1.319 | 0.045 |
| 4060358 | ABCA1 | NM_005502.2 | 1.307 | 0.00185 |
| 5420095 | MYC | NM_002467.3 | 1.302 | 0.00377 |
| 6960195 | LOC650646 | XM_942527.2 | 1.301 | 0.0432 |
| 3710397 | EFNA1 | NM_004428.2 | 1.251 | 0.0055 |
| 2570253 | BTN3A2 | NM_007047.3 | 1.246 | 0.0272 |
| 1940632 | NCAPG2 | NM_017760.5 | 1.238 | 0.0261 |
| 3800647 | UGCG | NM_003358.1 | 1.233 | 0.00373 |
| 6590377 | RPS26 | NM_001029.3 | 1.226 | 0.0457 |
| 5490408 | CEBPD | NM_005195.3 | 1.222 | 0.0436 |
| 3130296 | AMY2A | NM_000699.2 | 1.221 | 0.0485 |
| 160370 | TPM2 | NM_213674.1 | 1.209 | 0.0474 |
| 3130301 | PIM1 | NM_002648.2 | 1.203 | 0.0173 |
| 1440736 | LDLR | NM_000527.2 | 1.2 | 0.0263 |
| 6250010 | GPRIN3 | NM_198281.2 | 0.833 | 0.0147 |
| 1260086 | ID2 | NM_002166.4 | 0.814 | 0.0158 |
| 5890730 | RPS26L | XR_017804.1 | 0.805 | 0.0405 |

Gene List 6 - Uniquely deregulated in ACPA-pos RA vs OA

| Symbol | RefSeq | FC |
|---|---|---|
| HLA-A29.1 | NM_001080840.1 | 1.961 |
| C20orf100 | NM_032883.1 | 1.381 |
| IGFL2 | NM_001002915.1 | 1.381 |
| KIAA0101 | NM_014736.4 | 1.337 |
| TYMS | NM_001071.1 | 1.334 |
| CDCA7 | NM_031942.4 | 1.311 |
| CDCA5 | NM_080668.2 | 1.305 |
| UBE2C | NM_181800.1 | 1.278 |
| APOBEC3H | NM_181773.2 | 1.274 |
| LOC731682 | XM_001129369.1 | 1.274 |
| CDK5RAP3 | NM_176095.1 | 1.273 |
| TOP2A | NM_001067.2 | 1.269 |
| CCNB2 | NM_004701.2 | 1.258 |
| MGC4677 | NM_052871.3 | 1.239 |
| CDC20 | NM_001255.2 | 1.236 |
| CDC45L | NM_003504.3 | 1.236 |
| CRIP2 | NM_001312.2 | 1.235 |
| FKSG30 | NM_001017421.1 | 1.232 |
| CPA5 | NM_080385.3 | 1.231 |
| ICA1 | NM_004968.2 | 1.23 |
| NUSAP1 | NM_018454.5 | 1.23 |
| DDX11 | NM_030653.3 | 1.224 |
| CLEC2B | NM_005127.2 | 1.222 |
| MAF | NM_001031804.1 | 1.219 |
| PTTG1 | NM_004219.2 | 1.219 |
| GBP5 | NM_052942.2 | 1.218 |
| UHRF1 | NM_001048201.1 | 1.216 |
| FLJ33590 | NM_173821.1 | 1.21 |
| MUC1 | NM_001044391.1 | 1.21 |
| NLRC5 | NM_032206.3 | 1.21 |
| MT2A | NM_005953.2 | 1.209 |
| NCAPG | NM_022346.3 | 1.208 |
| CYorf15B | NM_032576.2 | 1.207 |
| MELK | NM_014791.2 | 1.207 |
| TK1 | NM_003258.2 | 1.207 |
| ATF4 | NM_001675.2 | 1.206 |
| HERPUD1 | NM_001010990.1 | 1.205 |
| ARPC5L | NM_030978.1 | 1.204 |
| ZNF655 | NM_001009957.1 | 1.203 |
| MYADM | NM_001020820.1 | 0.818 |
| FCGBP | NM_003890.1 | 0.688 |
| SERPINE2 | NM_006216.2 | 0.628 |
| PRKAR1A | NM_002734.3 | 0.522 |

Biological Functions: proportion of genes in a given gene list
assigned particular biological function is given, along with p-value . . .

| | “Cancer” |
|---|---|
| “Cell cycle” subset. | subset. |
| 24/43 (p < 10e−6) | 21/43 (p < 10e−6) |
| CCNB2 | CCNB2 |
| CDC20 | CDC20 |
| CDC45L | CDCA5 |
| CDCA5 | CDCA7 |
| CDCA7 | FCGBP |
| CDK5RAP3 (includes EG: 80279) | KIAA0101 |
| DDX11 | MAF |
| FCGBP | MELK |
| KIAA0101 | MT2A |
| MAF | MUC1 |
| MELK | NCAPG (includes |
| | EG: 64151) |
| MT2A | PRKAR1A |
| MUC1 | PTTG1 |
| NCAPG (includes EG: 64151) | SERPINE2 |
| NUSAP1 | TK1 |
| PRKAR1A | TOP2A |
| PTTG1 | TP53INP1 |
| SERPINE2 | TYMS |
| TK1 | UBE2C |
| TOP2A | UHRF1 |
| TYMS | UHRF1 |
| UBE2C | |
| UHRF1 | |
| ZNF655 | |

Gene List 7 - Uniquely deregulated in ACPA-neg RA vs OA

| SYMBOL | RefSeq | FC |
|---|---|---|
| HLA-DRB4 | NM_021983.4 | 1.701 |
| HBEGF | NM_001945.1 | 1.607 |
| MNDA | NM_002432.1 | 1.558 |
| LOC650298 | XM_939387.1 | 1.555 |
| CDKN1A | NM_000389.2 | 1.505 |
| CFD | NM_001928.2 | 1.499 |
| LOC644934 | XM_930344.2 | 1.476 |
| TNFSF10 | NM_003810.2 | 1.404 |
| C6orf105 | NM_032744.1 | 1.399 |
| GPR132 | NM_013345.2 | 1.399 |
| GSTM1 | NM_000561.2 | 1.394 |
| DACT1 | NM_016651.5 | 1.374 |
| GADD45A | NM_001924.2 | 1.373 |
| CSDA | NM_003651.3 | 1.369 |
| NCF1 | NM_000265.4 | 1.369 |
| BCL6 | NM_001706.2 | 1.363 |
| RPS26L | XR_017804.1 | 1.362 |
| AQP9 | NM_020980.2 | 1.349 |
| FAM46C | NM_017709.3 | 1.334 |
| IFNGR2 | NM_005534.2 | 1.329 |
| SLC7A5 | NM_003486.5 | 1.321 |
| F2RL1 | NM_005242.3 | 1.316 |
| ADM | NM_001124.1 | 1.313 |
| LOC653853 | XM_936029.1 | 1.309 |
| S100P | NM_005980.2 | 1.305 |
| AP3M2 | NM_006803.2 | 1.296 |
| CDKN2D | NM_001800.3 | 1.296 |
| RGS16 | NM_002928.2 | 1.295 |
| DDIT4 | NM_019058.2 | 1.291 |
| TMEM173 | NM_198282.1 | 1.287 |
| TMEM49 | NM_030938.2 | 1.284 |
| PRDX3 | NM_006793.2 | 1.282 |
| IFNGR1 | NM_000416.1 | 1.279 |
| GNG10 | NM_001017998.2 | 1.276 |
| UGP2 | NM_006759.3 | 1.271 |
| MT1E | NM_175617.3 | 1.27 |
| FKBP5 | NM_004117.2 | 1.269 |
| MSL3L1 | NM_078628.1 | 1.265 |
| SNORA10 | NR_002327.1 | 1.265 |
| FBXO5 | NM_012177.2 | 1.264 |
| GALNAC4S-6ST | NM_015892.2 | 1.262 |
| SLA | NM_001045556.1 | 1.262 |
| TMEM140 | NM_018295.2 | 1.261 |
| FURIN | NM_002569.2 | 1.259 |
| FAM113B | NM_138371.1 | 1.258 |
| GLRX | NM_002064.1 | 1.258 |
| CEBPB | NM_005194.2 | 1.257 |
| PDCL | NM_005388.3 | 1.253 |
| ELMO2 | NM_182764.1 | 1.252 |
| BAG3 | NM_004281.3 | 1.251 |
| HIF1A | NM_001530.2 | 1.251 |
| NFIL3 | NM_005384.2 | 1.251 |
| REXO2 | NM_015523.2 | 1.251 |
| RYBP | NM_012234.4 | 1.251 |
| GCA | NM_012198.2 | 1.249 |
| LY96 | NM_015364.2 | 1.249 |
| LOC145853 | XM_096885.9 | 1.247 |
| SLC20A1 | NM_005415.3 | 1.247 |
| ACVR2A | NM_001616.3 | 1.245 |
| LMNB1 | NM_005573.2 | 1.245 |
| SDCBP | NM_001007067.1 | 1.244 |
| DDX21 | NM_004728.2 | 1.243 |
| CLDN5 | NM_003277.2 | 1.241 |
| POLR1C | NM_203290.1 | 1.239 |
| GNL3 | NM_206826.1 | 1.237 |
| IL21R | NM_181078.1 | 1.237 |
| PIM2 | NM_006875.2 | 1.237 |
| SERPINB1 | NM_030666.2 | 1.236 |
| MXD1 | NM_002357.2 | 1.235 |
| CLECL1 | NM_172004.2 | 1.232 |
| NAMPT | NM_005746.2 | 1.232 |
| NDUFV2 | NM_021074.1 | 1.232 |
| ACVR1B | NM_020328.2 | 1.23 |
| FCGR1B | NM_001017986.1 | 1.229 |
| EIF4A3 | NM_014740.2 | 1.228 |
| GADD45B | NM_015675.2 | 1.227 |
| GRAMD4 | NM_015124.2 | 1.227 |
| EDA | NM_001005611.1 | 1.225 |
| PIM3 | XM_938171.2 | 1.225 |
| CSTF2T | NM_015235.2 | 1.224 |
| MAT2A | NM_005911.4 | 1.224 |
| BIRC3 | NM_001165.3 | 1.22 |
| LOC44035 | XM_496143.2 | 1.218 |
| NGRN | NM_016645.2 | 1.218 |
| SLC25A44 | NM_014655.1 | 1.218 |
| TRIB3 | NM_021158.3 | 1.217 |
| GNPDA1 | NM_005471.3 | 1.215 |
| SOD2 | NM_001024466.1 | 1.215 |
| PTGER2 | NM_000956.2 | 1.213 |
| LGALS8 | NM_006499.3 | 1.212 |
| SMAP2 | NM_022733.1 | 1.212 |
| GIMAP5 | NM_018384.3 | 1.211 |
| FAM89A | NM_198552.1 | 1.21 |
| GP5 | NM_004488.1 | 1.21 |
| METRNL | NM_001004431.1 | 1.21 |
| KISS1R | NM_032551.3 | 1.209 |
| FLJ43692 | NM_001003702.1 | 1.208 |
| ZNF828 | NM_032436.1 | 1.207 |
| HIAT1 | NM_033055.2 | 1.206 |
| RNF149 | NM_173647.2 | 1.205 |
| GIMAP7 | NM_153236.3 | 1.203 |
| TLR8 | NM_016610.2 | 1.203 |
| PTRH2 | NM_016077.3 | 1.202 |
| STS-1 | NM_032873.3 | 1.202 |
| IL2RA | NM_000417.1 | 1.2 |
| STAT3 | NM_139276.2 | 1.2 |
| DNAJB1 | NM_006145.1 | 0.831 |
| LY9 | NM_001033667.1 | 0.83 |
| XAF1 | NM_199139.1 | 0.827 |
| MAST3 | NM_015016.1 | 0.824 |
| C10orf73 | XM_096317.11 | 0.813 |
| FAM119B | NM_015433.2 | 0.811 |
| IL16 | NM_172217.2 | 0.806 |
| IFI27 | NM_005532.3 | 0.799 |

Gene List 8 Deregulated in (ACPA-neg AND ACPA-pos RA) vs OA

| | | ACPA-neg RA vs | ACPA-pos RA |
|---|---|---|---|
| Symbol | RefSeq | OA | vs OA |
| SOCS3 | NM_003955.3 | 1.916 | 1.452 |
| BCL3 | NM_005178.2 | 1.797 | 1.434 |
| SBNO2 | NM_014963.2 | 1.618 | 1.369 |
| BATF | NM_006399.2 | 1.594 | 1.372 |
| MTHFD2 | NM_001040409.1 | 1.561 | 1.332 |
| STX11 | NM_003764.2 | 1.553 | 1.311 |
| SOCS1 | NM_003745.1 | 1.49 | 1.328 |
| GPRIN3* | CR743148 | 1.457 | 1.331 |
| ARID5A | NM_212481.1 | 1.448 | 1.202 |
| TMEM70 | NM_017866.4 | 1.442 | 1.255 |
| ICOS | NM_012092.2 | 1.438 | 1.37 |
| LOC731186 | XM_001128760.1 | 1.435 | 1.272 |
| MIAT | NR_003491.1 | 1.428 | 1.546 |
| SOCS2 | NM_003877.3 | 1.403 | 1.233 |
| FAM100B | NM_182565.2 | 1.352 | 1.252 |
| FBXO32 | NM_058229.2 | 1.331 | 1.246 |
| SLC2A3 | NM_006931.1 | 1.329 | 1.203 |
| ST6GALNAC1 | NM_018414.2 | 1.329 | 1.256 |
| PRDM1 | NM_182907.1 | 1.328 | 1.335 |
| CEACAM1 | NM_001024912.1 | 1.321 | 1.236 |
| TRIB1 | NM_025195.2 | 1.316 | 1.224 |
| SFXN1 | NM_022754.4 | 1.312 | 1.225 |
| LDHA | NM_005566.1 | 1.302 | 1.215 |
| LINS1 | NM_181740.1 | 1.298 | 1.246 |
| NFKBIZ | NM_001005474.1 | 1.281 | 1.232 |
| SNORA64 | NR_002326.1 | 1.278 | 1.3 |
| CTLA4 | NM_005214.3 | 1.277 | 1.326 |
| B3GNT2 | NM_006577.5 | 1.274 | 1.216 |
| TLR6 | NM_006068.2 | 1.271 | 1.201 |
| TP53INP1 | NM_033285.2 | 1.266 | 1.278 |
| ARID5B | NM_032199.1 | 1.258 | 1.234 |
| NPDC1 | NM_015392.2 | 1.257 | 1.272 |
| P2RY5 | NM_005767.4 | 1.247 | 1.201 |
| PDCD1 | NM_005018.1 | 1.236 | 1.427 |
| SLC2A14 | NM_153449.2 | 1.211 | 1.21 |
| CCDC58 | NM_001017928.2 | 0.811 | 0.815 |
| NOG | NM_005450.2 | 0.779 | 0.802 |
| ID3 | NM_002167.2 | 0.75 | 0.757 |
| CD40LG | NM_000074.2 | 0.739 | 0.81 |
| LRRN3 | NM_001099660.1 | 0.578 | 0.679 |

Biological Functions: proportion of genes in a given gene list
assigned particular biological function is given, along with p-value . . .

| | T-lymphocyte | T-lymphocyte |
|---|---|---|
| Cell development | differentiation | development |
| 14/40 (p < 10e−10) | 7/40 (2.6e−7) | 9/40 (3.14e−7) |
| BATF | BCL3 | BCL3 |
| BCL3 | CD40LG | CD40LG |
| CD40LG | CTLA4 | CTLA4 |
| CEACAM1 | ICOS | ICOS |
| CTLA4 | ID3 | ID3 |
| ICOS | NOG | NOG |
| ID3 | SOCS3 | PDCD1 |
| NOG | | SOCS1 |
| PDCD1 | | SOCS3 |
| PRDM1 | | |
| SFXN1 | | |
| SOCS1 | | |
| SOCS2 | | |
| SOCS3 | | |

*transcript CR743148 (Illumine Probe ID 6370082) has been retired from NCBI, but the EST corresponds to splice variant(s) within the GPRIN3 gene (chromosome 4.90).

Gene List 9 Lists of Functionally-related Genes based on Pathway
analysis of Lists 5, 6 and 7 combined (n = 197)
(Uniquely de-regulated in RA vs OA, but not in inflammatory
controls)
Canonical Pathways. Proportion of genes listed in particular
pathway that appear is given in each case, along with p-value
for significance . . .

| T Helper Cell | | |
|---|---|---|
| Differentiation | Cell cycle | Interferon signalling |
| 6/41 (p = 2.63e−4) | (G2/M DNA damage | 3/30 (p = 1.8e−3) |
| | checkpoint regn) | |
| | 4/43(p = 3.25e−4) | |
| ICA1 | CCB2 | IFNGR1 |
| IFNGR1 | CDKN1A | IFNGR2 |
| IFNGR2 | GADD45A | SOCS1 |
| SOCS1 | TOP2A | |
| SOCS2 | | |
| SOCS3 | | |

| IL-9 signalling | JAK/STAT signalling |
|---|---|
| 3/37 (p = 2.44e−03) | 4/64 (p = 1.97e−03) |
| BCL3 | CDKN1A |
| SOCS2 | SOCS1 |
| SOCS3 | SOCS2 |
| | SOCS3 |

Biological Functions: proportion of genes in a given gene list
assigned particular biological function is given, along with p-value . . .

| | | | T-cell |
|---|---|---|---|
| Cell death | Cell Survival | Cell Proliferation | proliferation |
| 97/197 | 79/197 | 67/197 | 17/197 |
| (p = 2.31e−23) | (p = 2.97e−7) | (p = 2.28e−20) | (p = 2.27e−7) |
| ACVR1B | ACVR2A | ACVR2A | B3GNT2 |
| ACVR2A | ADM | ADM | BATF |
| ADM | AP3M2 | ARID5B | CD40LG |
| AP3M2 | ATF4 | ATF4 | CDKN1A |
| BAG3 | BCL3 | B3GNT2 | CEACAM1 |
| BCL3 | BCL6 | BATF | CLECL1 |
| BCL6 | CCNB2 | BCL3 | CTLA4 |
| BIRC3 | CD40LG | BCL6 | F2RL1 |
| CCNB2 | CDC20 | CD40LG | GADD45A |
| CD40LG | CDCA5 | CDC45L | ICOS |
| CDC20 | CDCA7 | CDCA7 | IFNGR1 |
| CDC45L | CDKN1A | CDKN1A | IL2RA |
| CDCA5 | CDKN2D | CDKN2D | PDCD1 |
| CDCA7 | CEACAM1 | CEACAM1 | PRDM1 |
| CDK5RAP3 (incl | CEBPB | CEBPB | SOCS1 |
| EG: 80279) | | | |
| CDKN1A | CFD | CLECL1 | SOCS3 |
| CDKN2D | CTLA4 | CRIP2 | TNFSF10 |
| CEACAM1 | DDIT4 | CSDA | |
| CEBPB | FCGBP | CTLA4 | |
| CFD | FKBP5 | DDX11 | |
| CSDA | FURIN | DDX21 | |
| CTLA4 | GADD45A | F2RL1 | |
| DDIT4 | GLRX | FKBP5 | |
| DNAJB1 | GPR132 | FURIN | |
| EDA | GSTM1 | GADD45A | |
| FBXO32 | HBEGF | GNL3 | |
| FCGBP | HERPUD1 | GPR132 | |
| FKBP5 | HIF1A | GSTM1 | |
| FURIN | HLA-DRB4 | HBEGF | |
| GADD45A | ICOS | HIF1A | |
| GIMAP5 | IFI27 | ICOS | |
| GLRX | IFNGR1 | ID3 | |
| GNL3 | IFNGR2 | IFNGR1 | |
| GPR132 | IL2RA | IL16 | |
| GSTM1 | KIAA0101 | IL21R | |
| HBEGF | LDHA | IL2RA | |
| HERPUD1 | LGALS8 | KIAA0101 | |
| HIF1A | MAF | KISS1R | |
| HLA-DRB4 | MAT2A | LDHA | |
| ICOS | MELK | LY96 | |
| ID3 | MT1E | MT2A | |
| IFI27 | MT2A | MUC1 | |
| IFNGR1 | MTHFD2 | MXD1 | |
| IFNGR2 | MUC1 | NAMPT | |
| IL2RA | MXD1 | NCF1 | |
| KIAA0101 | NAMPT | NOG | |
| LDHA | NCAPG (includes | NPDC1 | |
| | EG: 64151) | | |
| LGALS8 | NDUFV2 | PDCD1 | |
| LMNB1 | NFIL3 | PIM2 | |
| | | (includes | |
| | | EG: 11040) | |
| MAF | NOG | PRDM1 | |
| MAT2A | NPDC1 | PRDX3 | |
| MELK | PIM2 (includes | PRKAR1A | |
| | EG: 11040) | | |
| MT1E | PRDX3 | PTGER2 | |
| MT2A | PRKAR1A | PTTG1 | |
| MTHFD2 | PTGER2 | S100P | |
| MUC1 | PTTG1 | SERPINE2 | |
| MXD1 | S100P | SLC7A5 | |
| NAMPT | SDCBP | SOCS1 | |
| NCAPG (includes | SERPINB1 | SOCS2 | |
| EG: 64151) | | | |
| NCF1 | SERPINE2 | SOCS3 | |
| NDUFV2 | SLC2A3 | SOD2 | |
| NFIL3 | SLC2A14 | TNFSF10 | |
| NFKBIZ | SLC7A5 | TP53INP1 | |
| NOG | SOCS1 | TRIB1 | |
| NPDC1 | SOCS2 | TYMS | |
| PDCD1 | SOCS3 | UBE2C | |
| PIM2 (includes | SOD2 | UHRF1 | |
| EG: 11040) | | | |
| PRDM1 | TK1 | | |
| PRDX3 | TNFSF10 | | |
| PRKAR1A | TOP2A | | |
| PTGER2 | TP53INP1 | | |
| PTRH2 | TRIB1 | | |
| PTTG1 | TYMS | | |
| RYBP | UBE2C | | |
| S100P | UHRF1 | | |
| SDCBP | XAF1 | | |
| SERPINB1 | | | |
| SERPINE2 | | | |
| SLC2A3 | | | |
| SLC2A14 | | | |
| SLC7A5 | | | |
| SOCS1 | | | |
| SOCS2 | | | |
| SOCS3 | | | |
| SOD2 | | | |
| TK1 | | | |
| TLR6 | | | |
| TMEM173 | | | |
| TNFSF10 | | | |
| TOP2A | | | |
| TP53INP1 | | | |
| TRIB1 | | | |
| TRIB3 | | | |
| TYMS | | | |
| UBE2C | | | |
| UHRF1 | | | |
| XAF1 | | | |

| Blood cell differentiation | T-cell differentiation |
|---|---|
| 25/197 (p = 4.5e−12) | 15/197 (p = 3.3e−09) |
| ACVR1B | BCL3 |
| ACVR2A | BCL6 |
| BCL3 | CD40LG |
| BCL6 | CEBPB |
| CD40LG | CTLA4 |
| CDKN2D | GIMAP5 |
| CEBPB | ICOS |
| CTLA4 | ID3 |
| GIMAP5 | IFNGR2 |
| HIF1A | IL21R |
| ICOS | IL2RA |
| ID3 | MAF |
| IFNGR2 | MUC1 |
| IL21R | NOG |
| IL2RA | SOCS3 |
| MAF | |
| MUC1 | |
| NOG | |
| PDCD1 | |
| PRDM1 | |
| PRDX3 | |
| SFXN1 | |
| SOCS1 | |
| SOCS3 | |
| TNFSF10 | |

### REFERENCES

1. Klareskog L, Catrina A I, Paget S, Klareskog L, Catrina A I, Paget S. Rheumatoid arthritis. Lancet 2009; 373(9664):659-72.
2. Combe B, Landewe R, Lukas C, Bolosiu H D, Breedveld F, Dougados M, et al. EULAR recommendations for the management of early arthritis: report of a task force of the European Standing Committee for International Clinical Studies Including Therapeutics (ESCISIT). [see comment]. Annals of the Rheumatic Diseases 2007; 66(434-45.
3. van Gaalen F A, Linn-Rasker SP, van Venrooij W J, de Jong B A, Breedveld F C, Verweij CL, et al. Autoantibodies to cyclic citrullinated peptides predict progression to rheumatoid arthritis in patients with undifferentiated arthritis: a prospective cohort study. Arthritis & Rheumatism 2004; 50(3):709-15.
4. van Der Helm-van Mil A H M, Detert J, Cessie S L, Filer A, Bastian H, Burmester G R, et al. Validation of a prediction rule for disease outcome in patients with recent-onset undifferentiated arthritis: Moving toward individualized treatment decision-making. Arthritis & Rheumatism 2008; 58(8):2241-7.
5. Nishimura K, Sugiyama D, Kogata Y, Tsuji G, Nakazawa T, Kawano S, et al. Meta-analysis: diagnostic accuracy of anti-cyclic citrullinated peptide antibody and rheumatoid factor for rheumatoid arthritis. [see comment]. Annals of Internal Medicine 2007; 146(11):797-808.
6. Pratt A G, Isaacs J D, Wilson G. The clinical utility of a rule for predicting rheumatoid arthritis in patients with early undifferentiated arthritis: comment on the article by van der Helm-van Mil et al. [comment]. Arthritis & Rheumatism 2009; 60(3):905; author reply 906.
7. van't Veer L J, Bernards R, van't Veer L J, Bernards R. Enabling personalized cancer medicine through analysis of gene-expression patterns. Nature 2008; 452(7187):564-70.
8. Pascual V, Chaussabel D, Banchereau J. A genomic approach to human autoimmune diseases. Annual Review of Immunology 2010; 28:535-71.
9. Toonen E J, Barrera P, Radstake T R, van Riel P L, Scheffer H, Franke B, et al. Gene expression profiling in rheumatoid arthritis: current concepts and future directions. Annals of the Rheumatic Diseases 2008; 67(12):1663-9.
10. Lequerre T, Gauthier-Jauneau A-C, Bansard C, Derambure C, Hiron M, Vittecoq O, et al. Gene profiling in white blood cells predicts infliximab responsiveness in rheumatoid arthritis. Arthritis Research & Therapy 2006; 8(4):R 105.
11. McKinney E F, Lyons P A, Carr E J, Hollis J L, Jayne D R, Willcocks L C, et al. A CD8+ T cell transcription signature predicts prognosis in autoimmune disease. Nature Medicine 2010; 16(5):586-91.
12. van Baarsen L G M, Bos W H, Rustenburg F, van der Pouw Kraan T C T M, Wolbink G J J, Dijkmans B A C, et al. Gene expression profiling in autoantibody-positive patients with arthralgia predicts development of arthritis. Arthritis & Rheumatism 2010; 62(3):694-704.
13. Batliwalla F M, Baechler E C, Xiao X, Li W, Balasubramanian S, Khalili H, et al. Peripheral blood gene expression profiling in rheumatoid arthritis. Genes & Immunity 2005; 6(5):388-97.,
14. Lyons P A, Koukoulaki M, Hatton A, Doggett K, Woffendin H B, Chaudhry AN, et al. Microarray analysis of human leucocyte subsets: the advantages of positive selection and rapid purification. BMC Genomics 2007; 8:64.
15. Koetz K, Bryl E, Spickschen K, O'Fallon W M, Goronzy J J, Weyand C M. T cell homeostasis in patients with rheumatoid arthritis. Proceedings of the National Academy of Sciences of the United States of America 2000; 97(16):9203-8.
16. Ponchel F, Morgan A W, Bingham S J, Quinn M, Buch M, Verburg R J, et al. Dysregulated lymphocyte proliferation and differentiation in patients with rheumatoid arthritis. Blood 2002; 100(13):4550-6.
17. McInnes I B, O'Dell JR. State-of-the-art: rheumatoid arthritis. Annals of the Rheumatic Diseases 2010; 69(11):1898-906.
18. Arnett F C, Edworthy S M, Bloch D A, McShane D J, Fries J F, Cooper NS, et al. The American Rheumatism Association 1987 revised criteria for the classification of rheumatoid arthritis. Arthritis & Rheumatism 1988; 31(3):315-24.
19. Schroeder A, Mueller O, Stocker S, Salowsky R, Leiber M, Gassmann M, et al. The RIN: an RNA integrity number for assigning integrity values to RNA measurements. BMC Molecular Biology 2006; 7:3.
20. de Jager W, Prakken B J, Bijlsma J W J, Kuis W, Rijkers GT. Improved multiplex immunoassay performance in human plasma and synovial fluid following removal of interfering heterophilic antibodies. Journal of Immunological Methods 2005; 300(1-2):124-35.
21. Hueber W, Tomooka B H, Zhao X, Kidd B A, Drijfhout J W, Fries J F, et al. Proteomic analysis of secreted proteins in early rheumatoid arthritis: anti-citrulline autoreactivity is associated with up regulation of proinflammatory cytokines. Annals of the Rheumatic Diseases 2007; 66(6):712-9.
22. Livak K J, Schmittgen T D, Livak K J, Schmittgen T D. Analysis of relative gene expression data using real-time quantitative PCR and the 2(-Delta Delta C(T)) Method. Methods (Duluth) 2001; 25(4):402-8.
23. Johnson W E, Li C, Rabinovic A. Adjusting batch effects in microarray expression data using empirical Bayes methods. Biostatistics 2007; 8(1):118-27.
24. Du P, Kibbe W A, Lin S M. Iumi: a pipeline for processing Illumina microarray. Bioinformatics 2008; 24(13):1547-8.
25. Benjamini Y, Hochberg Y. Controlling the false discovery rate: a practical and powerful approach to multiple testing. Journal of the Royal Statistical Society. Series B (Methodological) 1995; 57(1):289-300.
26. Cortes C, Vapnik V. Support-vector networks. Machine Learning 1995; 20(3):273-97.
27. Owaki T, Asakawa M, Morishima N, Mizoguchi I, Fukai F, Takeda K, et al. STAT3 is indispensable to IL-27-mediated cell proliferation but not to IL-27-induced Th1 differentiation and suppression of proinflammatory cytokine production. Journal of Immunology 2008; 180(5):2903-11.
28. Starr R, Willson T A, Viney E M, Murray L J, Rayner J R, Jenkins B J, et al. A family of cytokine-inducible inhibitors of signalling. Nature 1997; 387(6636):917-21.
29. El Kasmi K C, Smith A M, Williams L, Neale G, Panopoulos A D, Watowich SS, et al. Cutting edge: A transcriptional repressor and corepressor induced by the STAT3-regulated anti-inflammatory signaling pathway. [erratum appears in J Immunol. 2008 Mar. 1; 180(5):3612 Note: Panopolous, Athanasia [corrected to Panopoulos, Athanasia D]]. Journal of Immunology 2007; 179(11):7215-9.
30. Brocke-Heidrich K, Ge B, Cvijic H, Pfeifer G, Loftier D, Henze C, et al. BCL3 is induced by IL-6 via Stat3 binding to intronic enhancer HS4 and represses its own transcription. Oncogene 2006; 25(55):7297-304.
31. Richard M, Louahed J, Demoulin J B, Renauld J C, Richard M, Louahed J, et al. Interleukin-9 regulates NF-kappaB activity through BCL3 gene induction. Blood 1999; 93(12):4318-27.
32. Gao J, McConnell M J, Yu B, Li J, Balko J M, Black EP, et al. MUC1 is a downstream target of STAT3 and regulates lung cancer cell survival and invasion. International Journal of Oncology 2009; 35(2):337-45.
33. Akaishi H, Takeda K, Kaisho T, Shineha R, Satomi S, Takeda J, et al. Defective IL-2-mediated IL-2 receptor alpha chain expression in Stat3-deficient T lymphocytes. International Immunology 1998; 10(11):1747-51.
34. Matikainen S, Sareneva T, Ronni T, Lehtonen A, Koskinen P J, Julkunen I, et al. Interferon-alpha activates multiple STAT proteins and upregulates proliferation-associated IL-2Ralpha, c-myc, and pim-1 genes in human T cells. Blood 1999; 93(6):1980-91.
35. Nichane M, Ren X, Bellefroid E J. Self-regulation of Stat3 activity coordinates cell-cycle progression and neural crest specification. EMBO Journal; 29(1):55-67.
36. Hirano T, Ishihara K, Hibi M, Hirano T, Ishihara K, Hibi M. Roles of STAT3 in mediating the cell growth, differentiation and survival signals relayed through the IL-6 family of cytokine receptors. Oncogene 2000; 19(21):2548-56.
37. Altman D G, Bland J M. Diagnostic tests 2: Predictive values. BMJ 1994; 309(6947):102.
38. Goronzy J J, Weyand CM. Rheumatoid arthritis. Immunological Reviews 2005; 204:55-73.
39. Schonland S O, Lopez C, Widmann T, Zimmer J, Bryl E, Goronzy J J, et al. Premature telomeric loss in rheumatoid arthritis is genetically determined and involves both myeloid and lymphoid cell lineages. Proceedings of the National Academy of Sciences of the United States of America 2003; 100(23):13471-6.
40. van der Helm-van Mil A H M, Verpoort K N, Breedveld F C, Huizinga T W J, Toes R E M, de Vries R R P. The HLA-DRB1 shared epitope alleles are primarily a risk factor for anti-cyclic citrullinated peptide antibodies and are not an independent risk factor for development of rheumatoid arthritis. Arthritis & Rheumatism 2006; 54(4):1117-21.
41. Kokkonen H, Soderstrom I, Rocklov J, Hallmans G, Lejon K, Rantapaa Dahlqvist S. Up-regulation of cytokines and chemokines predates the onset of rheumatoid arthritis. Arthritis & Rheumatism, 62(2):383-91,
42. Karlson E W, Chibnik L B, Tworoger S S, Lee I M, Buring J E, Shadick N A, et al. Biomarkers of inflammation and development of rheumatoid arthritis in women from two prospective cohort studies. Arthritis & Rheumatism 2009; 60(3):641-52.
43. Schindler C W. Series introduction. JAK-STAT signaling in human disease. Journal of Clinical Investigation 2002; 109(9):1133-7.
44. Fonseca J E, Santos M J, Canhao H, Choy E. Interleukin-6 as a key player in systemic inflammation and joint destruction. Autoimmunity Reviews 2009; 8(7):538-42.
45. Nowell M A, Williams A S, Carty S A, Scheller J, Hayes A J, Jones G W, et al. Therapeutic targeting of IL-6 trans signaling counteracts STAT3 control of experimental inflammatory arthritis. Journal of Immunology 2009; 182(1):613-22.
46. Eyles J L, Hickey M J, Norman M U, Croker B A, Roberts A W, Drake SF, et al. A key role for G-CSF-induced neutrophil production and trafficking during inflammatory arthritis. Blood 2008; 112(13):5193-201.
47. Rho Y H, Solus J, Sokka T, Oeser A, Chung C P, Gebretsadik T, et al. Adipocytokines are associated with radiographic joint damage in rheumatoid arthritis. Arthritis & Rheumatism 2009; 60(7):1906-14.
48. El Kasmi K C, Smith A M, Williams L, Neale G, Panopoulos A D, Watowich S S, et al. Cutting edge: A transcriptional repressor and corepressor induced by the STAT3-regulated anti-inflammatory signaling pathway. [Erratum appears in J Immunol. 2008 Mar. 1; 180(5):3612 Note: Panopolous, Athanasia [corrected to Panopoulos, Athanasia D]]. Journal of Immunology 2007; 179(11):7215-9.
49. Nishimoto N, Miyasaka N, Yamamoto K, Kawai S, Takeuchi T, Azuma J. Long-term safety and efficacy of tocilizumab, an anti-IL-6 receptor monoclonal antibody, in monotherapy, in patients with rheumatoid arthritis (the STREAM study): evidence of safety and efficacy in a 5-year extension study. Annals of the Rheumatic Diseases 2009; 68(10):1580-4.
50. Cohen S, Fleischmann R. Kinase inhibitors: a new approach to rheumatoid arthritis treatment. Current Opinion in Rheumatology 2010; 22(3):330-5.

1. A method of diagnosing Rheumatoid arthritis in a patient, the method comprising:
obtaining a sample from the patient; and
determining expression levels of one or more genes selected from the group consisting of
BCL3,
SOCS3,
PIM1,
SBNO2,
LDHA,
CMAH,
NOG,
PDCD1,
IGFL2,
LOC731186,
MUC1, and
GPRIN3; and
comparing said expression levels to reference expression levels, wherein a difference in expression of said one or more genes indicates an increased likelihood that the patient has Rheumatoid arthritis (RA).

2. A method as in claim 1, wherein the group further comprises the gene CD40LG.

3. A method as in claim 1, wherein the reference expression levels are representative of levels found in samples comprising cells from a patient who does not have Rheumatoid arthritis (RA).

4. A method as in claim 1, wherein the step of determining expression levels of one or more genes includes determining expression levels for all of the genes selected from the group consisting of:
BCL3,
SOCS3,
PIM1,
SBNO2,
LDHA,
CMAH,
NOG,
PDCD1,
IGFL2,
LOC731186,
MUC1, and
GPRIN3.

5. A method as in claim 4, wherein the group further comprises the gene CD40LG, and wherein expression levels are also determined for CD40LG.

6. A method for typing a sample from an individual classified as having undifferentiated arthritis, or suspected to suffer from rheumatoid arthritis, the method comprising:
obtaining a sample from the individual; and
determining expression levels of one or more genes selected from the group consisting of
BCL3,
SOCS3,
PIM1,
SBNO2,
LDHA,
CMAH,
NOG,
PDCD1,
IGFL2,
LOC731186,
MUC1, and
GPRIN3; and
typing said sample on the basis of the expression levels determined; wherein said typing provides prognostic information related to the risk that the individual has rheumatoid arthritis (RA).

7. A method as in claim 6, wherein the group further comprises the gene CD40LG.

8. A method as in claim 6, wherein the step of determining expression levels of one or more genes includes determining expression levels for all of the genes selected from the group consisting of:
BCL3,
SOCS3,
PIM1,
SBNO2,
LDHA,
CMAH,
NOG,
PDCD1,
IGFL2,
LOC731186,
MUC1, and
GPRIN3.

9. A method as in claim 1, wherein expression levels are determined by determining RNA levels.

10. A method as in claim 1, wherein the sample comprises CD4+ T cells.

11. A method as in claim 1, wherein the sample is peripheral whole blood.

12. A method as in claim 11, further comprising a step of separating CD4+ T cells from peripheral whole blood.

13. A method as in claim 10, further comprising a step of extracting RNA from the CD4+ T cells.

14. A method as in claim 1, further comprising the step of combining the results with the results of known prediction analysis.

15. A method as in claim 14, wherein the known prediction analysis is the Leiden prediction rule.

16. A method of diagnosing rheumatoid arthritis in a patient, the method comprising:
obtaining a blood sample from the patient; and
determining expression/mRNA levels of 12 or more genes selected from the group defined in GENE LIST 2; and
comparing said expression/mRNA levels to a set of reference expression/mRNA levels, wherein a difference in expression of said 12 or more genes indicates an increased likelihood that the patient has Rheumatoid arthritis.

17. A method of diagnosing Rheumatoid arthritis in a patient, the method comprising:
obtaining a blood sample from the patient; and
determining levels of Interleukin-6 (IL-6); and
comparing said levels to a set of reference IL-6 levels, wherein an difference in expression of IL-6 indicates an increased likelihood that the patient has Rheumatoid arthritis.

18. A method as in claim 17, wherein the results of the IL-6 expression analysis are combined with the results of known prediction analysis.

19. An array comprising (a) a substrate and (b) 12 or more different elements,
each element comprising at least one polynucleotide that binds to a specific mRNA transcript, said mRNA transcript being of a gene selected from the group defined in GENE LIST 2.

20. An array comprising (a) a substrate and (b) one or more different elements,
each element comprising at least one polynucleotide that binds to a specific mRNA transcript, said mRNA transcript being of a gene selected from the group comprising;
BCL3,
SOCS3,
PIM1.SBNO2,
LDHA,
CMAH,
NOG,
PDCD1,
IGFL2,
LOC731186,
MUC1, and
GPRIN3.

21. An array as in claim 20, wherein the group of genes further comprises CD40LG.

22. An array comprising (a) a substrate and (b) 12 elements, each element comprising at least one polynucleotide that binds to an mRNA transcript, said array comprising a binding element for the mRNA of each of the following group of genes:
BCL3,
SOCS3,
PIM1,
SBNO2,
LDHA,
CMAH,
NOG,
PDCD1,
IGFL2,
LOC731186,
MUC1, and
GPRIN3.

23. An array as in claim 22, further comprising a binding element for the mRNA of the CD40LG gene.

24. An array as in claim 19, wherein the substrate is a solid substrate.

25-32. (canceled)

33. A method as in claim 8, wherein the group further comprises the gene CD40LG.

34. A method as in claim 6, wherein expression levels are determined by determining RNA levels.

35. A method as in claim 6, wherein the sample comprises CD4+ T cells.

36. A method as in claim 6, wherein the sample is peripheral whole blood.

37. A method as in claim 36, further comprising a step of separating CD4+ T cells from peripheral whole blood.

38. A method as in claim 35, further comprising a step of extracting RNA from the CD4+ T cells.

39. A method as in claim 6, further comprising the step of combining the results with the results of known prediction analysis.

40. A method as in claim 39, wherein the known prediction analysis is the Leiden prediction rule.

41. An array as in claim 20, wherein the substrate is a solid substrate.

42. An array as in claim 22, wherein the substrate is a solid substrate.


SEQUENCE LISTING (13 sequences)

SEQ ID NO: 1 - DNA, 1813 bp, Homo sapiens
  ref <308>: DATABASE ACCESSION NUMBER: NM_005178.2
  ref <309>: DATABASE ENTRY DATE: 2007-01-28
  ref <313>: RELEVANT RESIDUES IN SEQ ID NO: (1)..(1813)
ccgtccccggcggccccatgccccgatgccccgcgggggccatggacgaggggcccgtggacctgcgcacccggcccaaggccgccggactcccgggcgccgcgctgccgctccgcaagcgcccgctgcgcgcgccctccccggagcccgccgctccccgcggcgctgcgggccttgtcgtccccctggaccctctgcgcggcggctgcgacctgccggcggtccccgggcccccccacggcctggcccggccggaggcgctttactaccccggagccttactgcctttgtaccccactcgggccatgggctccccgtttcctctggtgaacctgcctacacccctataccccatgatgtgccccatggaacaccccctttctgctgacatcgccatggccacccgtgcagatgaggacggagacacgcctctccatattgctgtggtgcagggtaacctgccagctgtgcaccggctggtcaacctcttccagcaggggggccgggagctcgacatctacaacaacctacggcagacaccgctccacctggctgtgatcaccacattaccgtctgtggtccggctcctggtgacagctggtgccagccccatggcgctggaccgccatggccagacggccgctcacctggcgtgcgagcaccgcagcccgacctgcctgcgagccctgctggacagcgcagctccgggcacgttggacctggaggcccgcaattatgacgggctcaccgccctgcacgtggcagtgaacaccgagtgccaagaaaccgtgcagctcttgctagagcgcggtgccgacatcgacgcagtggacattaagagcggccgctccccgctcatccacgccgtggaaaacaacagccttagcatggtgcagctgctgctgcagcacggcgccaacgtgaacgcgcaaatgtactccggcagctccgccctgcactcagcgtccggccgcgggctcctcccgctggtgcgcacgctggtccgcagcggcgctgacagcagcctcaagaactgccacaacgacacgccgctcatggtggcgcgcagccgcagggtcatcgacatcctgagggggaaggccacccggcctgcttccacctcccagccagacccctcccctgaccggagcgccaacacctcccccgagagcagcagccgcctcagctccaatggtcttctctccgcatcaccatcctcctcaccctcccagtctccccccagggacccccctggattccccatggctcctcccaatttcttccttccttccccatctccacccgccttcctgccctttgctggggtcctccgaggccctggccggccggtgcccccctccccagctccaggaggcagctgagggggatgggggggcagatcttggactcatgaggaggggcccccctgcccagaggggtcaacccttctggaaactgtgaagatctgacttcgcccccccccccccccatcttcgggaccaggatttgcacagaagcacatgcacctacccatacaccccctcttctgagcacagatgttcccccatctcgctccctcccaggactctgaccccagcattctcaggcaccagtccctgtccggaatgccacccacatcttccatttccatgtcccctcccagagctggtggacccagggaacagccactcccctccactctctaccagataactgaggaggggagaggtgggccgtaacgggcacggatcacgatgtaaattattaagcattttggttggatttcttttgtaataaactatttttgtaccat

SEQ ID NO: 2 - DNA, 2746 bp, Homo sapiens
ggctccgacttggactccctgctccgctgctgccgcttcggccccgcacgcagccagccgccagccgcccgcccggcccagctcccgccgcggccccttgccgcggtccctctcctggtcccctcccggttggtccgggggtgcgcagggggcagggcgggcgcccaggggaagctcgagggacgcgcgcgcgaaggctcctttgtggacttcacggccgccaacatctgggcgcagcgcgggccaccgctggccgtctcgccgccgcgtcgccttggggacccgagggggctcagccccaaggacggagacttcgattcgggaccagccccccgggatgcggtagcggccgctgtgcggaggccgcgaagcagctgcagccgccgccgcgcagatccacgctggctccgtgcgccatggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagtgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgcaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctttaaggggtaaagggcgcaaagggcatgggtcgggagaggggacgcaggcccctctcctccgtggcacatggcacaagcacaagaagccaaccaggagagagtcctgtagctctggggggaaagagggcggacaggcccctccctctgccctctccctgcagaatgtggcaggcggacctggaatgtgttggagggaagggggagtaccacctgagtctccagcttctccggaggagccagctgtcctggtgggacgatagcaaccacaagtggattctccttcaattcctcagcttcccctctgcctccaaacaggggacacttcgggaatgctgaactaatgagaactgccagggaatcttcaaactttccaacggaacttgtttgctctttgatttggtttaaacctgagctggttgtggagcctgggaaaggtggaagagagagaggtcctgagggccccagggctgcgggctggcgaaggaaatggtcacaccccccgcccaccccaggcgaggatcctggtgacatgctcctctccctggctccggggagaagggcttggggtgacctgaagggaaccatcctggtaccccacatcctctcctccgggacagtcaccgaaaacacaggttccaaagtctacctggtgcctgagagcccagggcccttcctccgttttaagggggaagcaacatttggaggggatggatgggctggtcagctggtctccttttcctactcatactataccttcctgtacctgggtggatggagcgggaggatggaggagacgggacatctttcacctcaggctcctggtagagaagacaggggattctactctgtgcctcctgactatgtctggctaagagattcgccttaaatgctccctgtcccatggagagggacccagcataggaaagccacatactcagcctggatgggtggagaggctgagggactcactggagggcaccaagccagcccacagccagggaagtggggagggggggcggaaacccatgcctcccagctgagcactgggaatgtcagcccagtaagtattggccagtcaggcgcctcgtggtcagagcagagccaccaggtcccactgccccgagccctgcacagccctccctcctgcctgggtgggggaggctggaggtcattggagaggctggactgctgccaccccgggtgctcccgctctgccatagcactgatcagtgacaatttacaggaatgtagcagcgatggaattacctggaacagttttttgtttttgtttttgtttttgtttttgtgggggggggcaactaaacaaacacaaagtattctgtgtcaggtattgggctggacagggcagttgtgtgttggggtggtttttttctctatttttttgtttgtttcttgttttttaataatgtttacaatctgcctcaatcactctgtcttttataaagattccacctccagtcctctctcctcccccctactcaggcccttgaggctattaggagatgcttgaagaactcaacaaaatcccaatccaagtcaaactttgcacatatttatatttatattcagaaaagaaacatttcagtaatttataataaagagcactattttttaatgaaaaaaaaaaaaaaaaa

SEQ ID NO: 3 - DNA, 2684 bp, Homo sapiens
cccgagaggagtcggtggcagcggcggcggcgggaccggcagcagcagcagcagcagcagcagcaaccactagcctcctgccccgcggcgctgccgcacgagccccacgagccgctcaccccgccgttctcagcgctgcccgaccccgctggcgcgccctcccgccgccagtcccggcagcgccctcagttgtcctccgactcgccctcggccttccgcgccagccgcagccacagccgcaacgccacccgcagccacagccacagccacagccccaggcatagccttcggcacagccccggctccggctcctgcggcagctcctctgggcaccgtccctgcgccgacatcctggaggttgggatgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatagcagcctttctggcaggtcctcccctctcttgtcagatgcccgagggaggggaagcttctgtctccagcttcccgagtaccagtgacacgtctcgccaagcaggacagtgcttgatacaggaacaacatttacaactcattccagatcccaggcccctggaggctgcctcccaacagtggggaagagtgactctccaggggtcctaggcctcaactcctcccatagatactctcttcttctcataggtgtccagcattgctggactctgaaatatcccgggggtggggggtgggggtgggtcagaaccctgccatggaactgttttcttcatcatgagttctgctgaatgccgcgatgggtcaggtaggggggaaacaggttgggatgggataggactagcaccattttaagtccctgtcacctcttccgactctttctgagtgccttctgtggggactccggctgtgctgggagaaatacttgaacttgcctcttttacctgctgcttctccaaaaatctgcctgggttttgttccctatttttctctcctgtcctccctcaccccctccttcatatgaaaggtgccatggaagaggctacagggccaaacgctgagccacctgcccttttttctgcctcctttagtaaaactccgagtgaactggtcttcctttttggtttttacttaactgtttcaaagccaagacctcacacacacaaaaaatgcacaaacaatgcaatcaacagaaaagctgtaaatgtgtgtacagttggcatggtagtatacaaaaagattgtagtggatctaatttttaagaaattttgcctttaagttattttacctgtttttgtttcttgttttgaaagatgcgcattctaacctggaggtcaatgttatgtatttatttatttatttatttggttcccttcctattccaagcttccatagctgctgccctagttttctttcctcctttcctcctctgacttggggaccttttgggggagggctgcgacgcttgctctgtttgtggggtgacgggactcaggcgggacagtgctgcagctccctggcttctgtggggcccctcacctacttacccaggtgggtcccggctctgtgggtgatggggaggggcattgctgactgtgtatataggataattatgaaaagcagttctggatggtgtgccttccagatcctctctggggctgtgttttgagcagcaggtagcctgctggttttatctgagtgaaatactgtacaggggaataaaagagatcttatttttttttttatacttggcgttttttgaataaaaaccttttgtcttaaaaaaaaaaaaaaaaaaaaaaaaaaaaaaaaaaaaaaaaaaaa

SEQ ID NO: 4 - DNA, 4948 bp, Homo sapiens
gtgcgcggggcccccaagatggcgcccgaaacccggaagtgagcggcggcagctgcgaggctcggagaaacaggcgccgcgggctccgcgcccggccggacccgggcccgagatcatgatgctgccgccaccgccgccaccacggagcgagaagcccagatagacgccccggcggccccgggtcctggagtcccgccgcctgctgcccggccgaggaccccaccccgcctgccgcccgatgcttgcagtggggcccgccatggacagggattacccgcagcatgaacccccgccggcgggcagcctcctgtacagcccgccgcccctgcagagcgccatgctgcactgcccctactggaacaccttctcgctgccgccataccctgccttctccagcgacagccgcccgttcatgagctccgcctccttcctcggcagccagccctgcccagacaccagctatgcccccgtggccaccgcctccagcttgccaccaaagacctgcgactttgctcaggactcctcctattttgaggacttctccaacatctccatcttctcctcgtccgtggactccctgtcggacatcgtggacacgcccgacttcctgccggctgacagcctcaaccaggtgtccaccatctgggacgataaccctgccccctccacccacgataagctgttccagctcagcaggccgtttgcaggcttcgaggactttctgccctcccacagcaccccgcttctcgtcagctaccaggagcagagtgtgcagagccagccagaggaggaggacgaggctgaggaggaggaggcggaggagctggggcacacagagacctacgccgactacgtgccgtccaagtccaagatcgggaagcagcacccagaccgcgtggtggagaccagcacactgtccagcgtcccacccccagacatcacctacaccctggccctgccctcggacagcggggccctgtctgccctgcagctagaggccatcacctacgcctgccagcaacacgaggtcctgctccccagcgggcagcgcgcgggctttctcatcggcgatggggccggcgtgggcaaaggccggacggtggccggagtcatcctggagaaccacctgcgcggccggaagaaagcattgtggttcagcgtctccaacgacctcaagtacgatgcggagcgcgacctgcgggacatcgaagccacgggcatcgcggtgcacgcgctcagcaagatcaagtacggtgacaccactacctcagagggcgtcctcttcgccacctactccgccctgattggggagagccaggccggcggccagcaccgcactcgcctccggcagatcctggactggtgtggggaggccttcgagggcgtcatcgtgttcgacgagtgtcacaaagccaagaatgccggctccaccaagatgggcaaggctgtgctagacctgcagaacaagctgcccctggcccgcgtggtctacgccagcgccacaggtgcctctgagcctcggaacatgatctacatgagccgcttgggtatctggggcgagggcacacccttccggaactttgaggagttcctgcacgccatcgagaagaggggcgttggcgccatggagatcgtggccatggacatgaaggtcagcggcatgtacatcgcacgccagctcagcttctccggcgtcaccttccgcatcgaggagatcccgctggccccagccttcgagtgcgtctacaaccgcgcggccctgctgtgggccgaggccctgaacgtgttccagcaggcggccgactggatcggcctggagtcgcgcaagtccctgtggggccagttctggtcggcacaccagcgcttcttcaagtatctgtgcatcgcagccaaggtgcgccggctggtggagctggcccgagaggagctggcgcgagacaagtgcgtggtcatcgggctgcagtccacgggcgaggcgcgcacgcgggaggtgctgggggagaacgatgggcacctcaactgcttcgtctcggccgctgaaggcgtgttcctgtcgctaattcagaagcactttccgtccaccaagagaaagcgggacagaggagcgggcagcaagcggaaacggcgacctcggggacgcggggccaaagccccccggctggcgtgcgagacagcgggcgtcatccgcatcagtgacgacagcagcacggagtcggaccctggcctggacagcgacttcaactcctcccccgagtccctggtggatgacgacgttgtcatcgttgatgcagtcgggctccccagtgacgaccggggacccctgtgcctcctgcagagagacccgcatggccccggggtcctggagcgggtggagcggctgaagcaggatctgctggacaaagtgcggcggctgggccgggaactgccagtcaacaccctggacgagctcatcgaccagctgggcggcccccagcgggtggcggagatgaccggcaggaaaggccgcgtggtgtccaggcccgacgggacggtggccttcgagtcgcgggcagagcagggtctgtccatcgaccacgtgaacctcagggagaagcagcgcttcatgagcggcgagaagctcgtggccatcatctcggaggcctccagctcgggtgtctccctccaagccgaccgccgtgtccagaaccagcggcgccgcgtgcacatgaccttggagctgccgtggagcgccgaccgcgccatccagcagttcggccgcacccaccggtccaaccaggtctccgcgccagagtatgtcttcctcatctcggagctggccggggagcgccggttcgcctccatcgtggccaagcgcctggagagtctgggggccctgacccacggagaccgccgcgccacggagtcccgtgacctcagcaagtacaactttgagaacaagtatggcacccgggccctgcactgtgtcctcaccaccatcctgagccagactgagaacaaagtgcctgtgccccagggataccctggaggggtccccaccttcttccgggacatgaagcagggcctgctgtctgtgggcattggtggccgggagtcccggaatggctgcctggacgtggagaaggactgttccatcaccaagttcctgaaccgcatcctggggctggaggtgcacaagcagaacgccctgttccagtacttctcagacaccttcgaccacctcatcgagatggacaagcgggagggcaaatacgacatgggcatcctggaccttgctcccggtatcgaggagatctacgaggagagccagcaggtgttcctggctcccgggcacccgcaggacgggcaggtggtcttctacaagatcagcgtggaccgcggcctgaagtgggaggacgcctttgccaagtcgctggcgctgacgggcccctatgacggcttctacctctcctacaaggtccgcggtaacaagcccagctgcctgctggcggagcagaaccgcggccagttcttcacggtgtacaagcccaacatcggccggcagagccagctggaggccctggacagcctccgccgcaagttccaccgggtcaccgcggaggaggccaaggagccctgggagagtggctacgctttgtcgctgacgcactgcagccacagcgcctggaaccggcactgccggctggcgcaggagggtaaggactgcctgcaggggctgcggctgcggcaccactacatgctgtgcggcgcgctgctgcgcgtgtggggccgcatcgccgccgtcatggccgacgtcagcagcagcagctacctgcagatcgtgcggctgaagaccaaggacaggaagaagcaagtgggcatcaagatccccgagggctgcgtgcgccgggtgctgcaggagctgcggctgatggatgcggacgtgaagcgcaggcaggcgcccgccctgggctgccccgccccgcccgccccgcgcccgctggcgctgccttgcggccccggagaggtgctggacctcacctacagccccccggccgaggccttcccgccgcccccgcacttctctttcccggcgccgctgtccctggacgccggccccggcgtcgtgccgctgggcacccccgacgcccaggccgaccctgcggccctcgcgcaccagggctgcgacatcaacttcaaggaggtgctggaggacatgctgcgctcgctgcacgcggggccgccctccgagggcgcgctgggggagggcgcgggggcggggggcgcggcgggcggtggtcccgagcggcagagcgtgatccagttcagcccacccttccccggcgcccaggctcctctctgacacgcctttaggcgaaacatgccccaagacacagggaccgtttctcccctaggagcagcggtggggagcagggccaaggtcccctgaccactgctcagaggagccctaggccctggccgcagtgccttcagcgcccgacccgggcccccacctggtcagccctggcggggcccactcaggacagctgggggccggggcgtggcagggccctctctgtgcctctcctcccaagtaggaaggggctccgggtggctgctctgggactgggcacccacaagggctcagtgggcccaaacccttgaaatccgtgaaaccgggtggtcccaagagctagaaactcaggaaaccccaggtgctcagggccccgcgtctcgggggctccgtggggcagacccctgctaatatatgcaattctccctcccccagcccttccctgacccctaagttattgcccgctcacctctcccaggccccaggccgcggagctggcagggtggcgcctgcggtttctatgtatttatagcaagttctgatgtacatatgtaaaggacttttttaaatatatgtgccttttgcctacttcccaaaaaaaaaaaaaaaaaaaaaaaa

SEQ ID NO: 5 - DNA, 1661 bp, Homo sapiens
tgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtg

SEQ ID NO: 6 - DNA, 2484 bp, Homo sapiens
catcgaacaaacaactgagatcctgttgtgcctatcacctgttgaagttgccagtcttaaggaaggaatcaatttctttcgcaataagagcactggcaaagactacgtcttgtacaagaataagagccgactgagggcatgcaagaatatgtgcaagcaccaaggaggcctgttcataaaagatatcgaggatttagccggaagttgttgaaatggatgaaaacaacggacttttgcttttagaactgaatcctcctaacccttgggacttacagcccagatctcctgaagagttggcttttggagaagtacagataacatatctcactcatgcctgcatggacctcaagttaggagacaagagaatggtgtttgacccttggttaatcggtcctgcttttgcccgtggatggtggttgctccatgagcctccatctgattggctggagaggctgtgccaggcagacctcatttacatcagtcatctgcactcagaccacctgagttaccccacactgaaaaagcttgctgggagaagaccagatattcccatttatgttggaaacacagaaaggcctgtattttggaatctgaatcagagcggtgtccagttgactaatatcaatgtcgtgccatttggaatatggcagcaggtggacaaaaatcttcgattcatgatcttgatggacggtgttcatcctgagatggacacttgcattattgtggagtacaaaggtcataaaatactcaatatagtagactgcaccagacccaatgggggaaggctgcctatgaaggttgctctaatgatgagtgattttgctggaggagcatcaggctttccaatgactttcagtggtggaaaatttacggaggaatggaaagcccaattcattaaaacagaaaggaagaagctcctgaactacaaggctcggctggtgaagaacctgcaaccccgaatttattgtccctttgctgggtattttgtggaatctcacccatcagacaagtacatcaaggaaacaaacaccaaaaatgacccaaatgaactcaacaatcttatcaagaaaaactctgatgtgataacatggacccctcgaccgggagccacccttgatctgggaagaatgctgaaggatcgaacagacagcaagggcatcatagagcctccagaggggacaaaaatttacaaggattcctgggattttgaaccttatttggaaatcttgaatgctgctctaggagatgaaatatttcttcactcatcctggataaaagaatacttcacttgggctggatttaaagattacaaccttgtggtcaggatgattgagacagatgaggacttcaatccttttcctggaggatatgactatttggttgactttttagatttatccttcccaaaagaaagaccacaacgagaacatccctatgaggaaatccatagccgggtggatgtcatcagacacgtggtgaagaatggtctactctgggatgagttgtatataggattccaaacacggctccagcgggatcctgacatataccatcacctgttttggaatcattttcaaataaaactccccctcacaccacccaactggaagtcattcctgatgtgctgtgagcagaatgggcctgtgattttgcaagaatgcaaaaccacatgaaaatttcaagaattcactgatctgatgcaaaataaaaatttatcattacatcttgaacccaggaagcttacagcaaagagactatgctttatgacgtcagcaatagataattccacgttgcctttgtgatttgtatatatagcttacatttgtggatcactacatagccagattcaaaaatattttacttgttccatccacagttctctacagaaagaaccaatgaacccaataggaacaaattctctgtggaaaacaaagcatagctgtagtagatacgaatccaatcacagaggaaacaggaagagaaaaacatccaagactacagtgaaaactggaaatggtctgttttcgtgatattcgtatgattaagatgcaaattttttcttaggaaaatgtgattgttaactagcattctgttttacatgttgacatttctaacacacacaccactgatttgaacttcaaaatttattttctgattatatatgctaggtctgattctgaagatacaagaattcaatggtggaatttgtctcctgaaatttctagattcataattagaagtgtttattaccaacttctcacttagattaacatcagtgatgtttgattattctggttaaccgctcacatgcataacaataatgctagaaattcaggaattataattttgtgatttatactgtgtggtaaattattcagttctcactgcaaataaatataatgtatatcaatgtaatgtaggtcagaatgtctatggtgagcaaataaaagattattttaaaatatgaaaaaaaaaaaaaaaaaa

SEQ ID NO: 7 - DNA, 1279 bp, Homo sapiens
cagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcgatccccaccgacgccccctgcaccgcctccaaccagttccaccaccctctagcgagggttttcaatgaacttttttttttttttttttttttttctgggctacagagacctagctttctggttcctgtaatgcactgtttaactgtgtaggaatgtatatgtgtgtgtatatacggtcccagttttaatttacttattaaaaggtcagtattatacgttaaaaaaaaaaaa

SEQ ID NO: 8 - DNA, 2106 bp, Homo sapiens
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttcttcccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagccgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctcccacctttacacatgcccaggcagcacctcaggccctttgtggggcagggaagctgaggcagtaagcgggcaggcagagctggaggcctttcaggccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgcgtggcctatccactcctcacatccacacactgcacccccactcctggggcagggccaccagcatccaggcggccagcaggcacctgagtggctgggacaagggatcccccttccctgtggttctattatattataattataattaaatatgagagcatgct

SEQ ID NO: 9 - DNA, 894 bp, Homo sapiens
tttgggtttggacgggtgcgtttgtttttcatagtgcaggtctggagccggactgagatggggagtagagggaccccttgaaacttcagggtggatgaatatgaagtttcatcaccagctgtgatgtttgatattggactgtggttgtgccagttgacactcaggagtggagactagatctgcaatctgttgggactgtgatgaggggatcatcctgccctcgaaccagacccagccctgtagcccaggcactattgcctggagttcctgggctctcagcgccaggaaatcatgaggttcagtgtctcaggcatgaggaccgactaccccaggagtgtgctggctcctgcttatgtgtcagtctgtctcctcctcttgtgtccaagggaagtcatcgctcccgctggctcagaaccatggctgtgccagccggcacccaggtgtggagacaagatctacaaccccttggagcagtgctgttacaatgacgccatcgtgtccctgagcgagacccgccaatgtggtcccccctgcaccttctggccctgctttgagctctgctgtcttgattcctttggcctcacaaacgattttgttgtgaagctgaaggttcagggtgtgaattcccagtgccactcatctcccatctccagtaaatgtgaaagcagaagacgttttccctgagaagacatagaaagaaaatcaactttcactaaggcatctcagaaacataggctagggtaatatgtgtaccagtagagaagcctgaggaatttacaaaatgatgcagctccaagccattgtatggcccatgtgggagactgatgggacatggagaatgacagtagattatcaggaaataaataaagtggtttttccaatgtaaaaaaaaaa

SEQ ID NO: 10 - DNA, 1095 bp, Homo sapiens
atgggttcagcccaaggcttggcatattgtggatgcttcgtagaagttattgtgggcttactgtcagctgtgcacaaggcaccaggagaacaggggctggaggtaggagagatgttgccagactgtcagttacttcttccagttagcttttcagttgtgttgcagtatgcctctggaggaggggaagtgtctttgatgctctggaatacaaccgggcccgcccccggcgcaccggcccgcgcgggcacctggcggtcgcagcggacacagcaggaggaggataggccgcgctgggcgggcgtcgcggggccgcgcttggtgttcctgagccgcggccgaactttttcccaagaagtcccacgtttggaggcccgaggagcgggacggggaggagagactggagttcctggaaggagcagcagcagcagccccacaacatggcctcctccgcctcctcctctcccgcgggcaccgaggactccgctcctgcccagggtggctttggatcagattattcacgaagctctagaaaggtaagtgaacttggcattggccattgcttattatgcttcctgtctggctcttactcatgttgccattttgtggactaaaactcaaggcagttcaaaaatactgatatgcaggtgccatggatgctgttggagtgtgtgtcctaagagagagggtaagtgctggttttccattttaaacatagctcgtgatatcaagttgccccctcccttcattccctgactcccgtcttcctccaaaagcggctctcttggtttagtaagcatctctaccctgcaaagcggaaataatgaaaaatgagattcactttgcaagcttgttgggtgagcttctggagaataatttttaccaaaagcctgaaatcatttggcattgttggtgttttcaatgcatctgcatcacagcacattgcatttcacatctggccagtttggaatgaaacttagaaaatcaccagactatagtgatgtaattgtcatgaagtggtttttgtagcttagagaaaagaaaaacacttggctgaaagaatggtctgaagtcccacccaaagtaaaacattctaaa

SEQ ID NO: 11 - DNA, 846 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaaa

SEQ ID NO: 12 - DNA, 730 bp, Homo sapiens
  misc_feature (728)..(728): n is a, c, g, t or u
attttaacagcaatgagatgacatgagaatatcttcaattcatcaaggatctgtaaagaaggagaggtacaagatatatgaaacccaaatctcaaaacaatgatttagtgaatttcccatgaactttaaacagtgattgcttcaaaatttccaagagccatactctccctccagctgctgtgtgtgtgtgtgtgtataaatgcacactattttaacctaaaatggtgccctgtggctgccattctctaactcttgcatacttaaacatttattcttggtcaaattaaaacctcatgcatttccaaagatataaatgccttgcctggagaagttagatcttgcaagtctcaggagggccgagatggtttgtcttatgcctatagctgtttatgtcccaccagtgggtgtttgtttcattaggtgccgtttccagccaaatgttctcattcttcacatcttcaatgttgagtagcaaacagaagaacatccttcttagcataatattgcttcactggactgattgtgaactcaaaatacctcttgtttcttgtgaagggtttgccttttgtaaacaatataagatcacttttggtcaaccaccctgtctgaatttatctgggctgctataataaagtatcataaactgggaggcttgtaaataaatttatttcttacagttttggaaagctgggaaccccaaaatcaaggccaatttggtgtctggtgangg

SEQ ID NO: 13 - DNA, 1834 bp, Homo sapiens
actttgacagtcttctcatgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaa